(19) United States
(12) Patent Application Publication
(10) Pub. No.: US 2026/0060565 A1
(43) Pub. Date: Mar. 5, 2026

(54) GASTROPARESIS DIAGNOSTIC METHOD, PROGRAM, AND APPARATUS

(71) Applicant: Atmo Biosciences Limited, Box Hill (AU)

(72) Inventors: James JOHN, Box Hill (AU); Malcolm HEBBLEWHITE, Box Hill (AU); Kyle BEREAN, Box Hill (AU); Adam CHRIMES, Box Hill (AU)

(21) Appl. No.: 19/105,635

(22) PCT Filed: Aug. 22, 2023
(86) PCT No.: PCT/AU2023/050803

§ 371 Date: Feb. 21, 2025

(30) Foreign Application Priority Data
Aug. 23, 2022 (AU) 2022902416

(51) Int. Cl. A61B 5/07; A61B 5/00

(52) U.S. Cl. CPC A61B 5/073; A61B 5/4255; A61B 5/7275; A61B 2560/0214; A61B 2560/0223; A61B 2560/0252; A61B 2560/0462; A61B 2562/0219; A61B 2562/0271; A61B 2562/029

(57) ABSTRACT

Embodiments include a method of diagnosing gastroparesis or suspected gastroparesis, the method comprising: obtaining data representing a time series of readings from gas sensing apparatus housed within a ingestible capsule device orally ingested by a subjected; processing the readings to detect one or more gastroparesis indicator spikes in the CO2 concentration with respect to time; based on the detected one or more gastroparesis indicator spikes in the CO2 concentration with respect to time, diagnosing gastroparesis or suspected gastroparesis.

### TECHNICAL FIELD

Embodiments are in the field of medical diagnosis and in particular relate to the diagnosis or detection of gastroparesis, or suspected gastroparesis, based on data obtained by sensors onboard an ingestible capsule device.

### INTRODUCTION

Gastroparesis is slowed down or non-existent stomach motility, causing delayed gastric emptying. Gastroparesis is associated with problems with blood sugar levels, nutritional issues, nausea, vomiting, and abdominal pain.

Present modes of diagnosis comprise one or a combination of:
scintigraphy: consumption of foodstuff containing radioactive material followed by radioactive scanning of a subject abdomen to monitor motility within the stomach and gastrointestinal tract;
breath tests: consumption of foodstuff followed by periodical collection of breath samples, concentration of gases in expired breath may indicate progress of foodstuff through gastrointestinal tract;
upper gastrointestinal endoscopy: a camera on the end of a tube is inserted into the GI tract via the mouth to provide information on stomach motility;
ultrasound and other non-invasive imaging techniques: imaging a subject abdomen can provide information regarding stomach motility.

Present modes of diagnosis are often indirect (such as breath tests) and therefore suffer from inaccuracy. Consumption of radioactive material is undesirable. Endoscopy is very uncomfortable for a subject. Imaging techniques provide only snapshots, and the nature of gastroparesis is that it requires observation over a long period of time to enable a positive diagnosis. Therefore, subject suffers inconvenience, cost, and resource usage associated with a prolonged period of time at a clinic at which imaging is performed.

It is desirable to at least somewhat ameliorate one or more issues associated with the existing techniques, or to provide a new alternative.

### STATEMENTS

Embodiments may include a method of diagnosing gastroparesis or suspected gastroparesis, the method comprising: obtaining data representing a time series of readings from gas sensing apparatus housed within a ingestible capsule device orally ingested by a subjected, the time series of readings being taken during exposure of the gas sensing apparatus to a gas mixture at the ingestible capsule device during passage of the ingestible capsule device through a gastrointestinal tract of the subject, each reading having a value, the values of the readings being sensitive to CO2 concentration in the gas mixture; processing the readings to detect one or more gastroparesis indicator spikes in the CO2 concentration with respect to time, a gastroparesis indicator spike being a spike in the CO2 concentration with respect to time at a timing after an ingestion timing of the ingestible capsule device and preceding a gastric-duodenal transition timing of the ingestible capsule device; based on the detected one or more gastroparesis indicator spikes in the CO2 concentration with respect to time, diagnosing gastroparesis or suspected gastroparesis.

Optionally, the gas sensing apparatus includes a thermal conductivity detector, TCD, gas sensor, wherein processing the readings from the subset to detect one or more spikes in the CO2 concentration values with respect to time includes referencing calibration data transforming TCD gas sensor reading values to CO2 concentration values.

Optionally, at a microcontroller or processor on the ingestible capsule device, controlling the TCD gas sensor to multiple operating temperature set points at which to make readings, wherein processing the readings from the subset to detect one or more gastroparesis indicator spikes in the CO2 concentration includes comparing the TCD gas sensor readings at different operating temperature set points with one another to calculate CO2 concentration values.

Optionally, diagnosing gastroparesis or suspected gastroparesis based on the detected one or more gastroparesis indicator spikes in the CO2 concentration with respect to time comprises: predicting presence or absence of gastroparesis in the subject by calculating values of each of one or more factors and inputting the calculated values to a predefined model outputting a quantitative or qualitative indication of likelihood of gastroparesis being present in the subject based on the one or more input calculated values; the calculated values of each of one or more factors comprising one or more from among: a count of the number of gastroparesis indicator spikes; a count of the number of gastroparesis indicator spikes and the height of each gastroparesis indicator spike; a histogram or another representation of distribution of heights of each gastroparesis indicator spike; an aggregate height of the gastroparesis indicator spikes;
a count of the number of gastroparesis indicator spikes and the area under each gastroparesis indicator spike; a histogram or another representation of distribution of areas under each gastroparesis indicator spike; an aggregate area under the gastroparesis indicator spikes; temporal duration between ingestion of the ingestible capsule device and the gastric-duodenal transition timing of the ingestible capsule device.

Optionally, the output of the predefined model includes an indication of severity of gastroparesis in the subject.

Optionally, the ingestible capsule device also houses an environmental temperature sensor to detect an environmental temperature at the ingestible capsule device, wherein the method further comprises preprocessing the readings from the gas sensing apparatus to compensate for changes in the environmental temperature.

Optionally, processing the readings to detect one or more gastroparesis indicator spikes, comprises: detecting a gastric-duodenal transition indicator in the time series of readings from the gas sensing apparatus, determining that the gastric-duodenal transition indicator in the time series of readings from the gas sensing apparatus is caused by a gastric-duodenal transition by the ingestible capsule device, and determining a timing of the gastric-duodenal transition indicator as the gastric-duodenal transition timing.

Optionally, processing the readings to detect one or more gastroparesis indicator spikes comprises: determining an upper bound on the gastric-duodenal transition timing by positively detecting residence of the ingestible capsule device in the intestines of the gastrointestinal tract;
detecting a chronologically latest spike in the CO2 concentration with respect to time preceding the upper bound as a gastric-duodenal transition indicator spike and determining that the gastric-duodenal transition indicator spike is caused by a gastric-duodenal transition by the ingestible capsule device, and determining a timing of the gastric-duodenal transition indicator as the gastric-duodenal transition timing.

Optionally, positively detecting residence of the ingestible capsule device in the intestines of the gastrointestinal tract includes detecting an ileocecal junction transition indicator, determining that the ileocecal junction indicator is caused by an ileocecal junction transition of the ingestible capsule device, and determining a timing of the ileocecal junction transition indicator as the upper bound on the gastric-duodenal indicator timing.

Optionally, the gas sensing apparatus includes a VOC gas sensor and the ileocecal junction transition indicator is a feature in a time series of readings from the VOC gas sensor, the reading being a turning point, a step change, or a period of gradient increase exceeding a gradient increase threshold.

Optionally, embodiments include obtaining data representing a time series of readings from a reflectometer housed within the ingestible capsule device and formed of an antenna in series with a directional coupler; determining the gastric-duodenal transition timing by: detecting a gastric-duodenal transition indicator in the time series of readings from the reflectometer, determining that the gastric-duodenal transition indicator in the time series of readings from the reflectometer is caused by a gastric-duodenal transition by the ingestible capsule device, and determining the gastric-duodenal transition timing based on the timing of the detected gastric-duodenal transition indicator.

Optionally, embodiments include obtaining data representing a time series of readings from an accelerometer housed within the ingestible capsule device; determining the gastric-duodenal transition timing by: detecting a gastric-duodenal transition indicator in the time series of readings from the accelerometer, determining that the gastric-duodenal transition indicator is caused by a gastric-duodenal transition by the ingestible capsule device, and determining the gastric-duodenal transition timing based on the timing of the detected gastric-duodenal transition indicator.

Optionally, a spike in the CO2 concentration with respect to time, being a gastroparesis indicator spike or a gastric-duodenal transition indicator spike, is detected by identifying: a first period of increasing CO2 concentration at a rate of change with respect to time exceeding an increase gradient threshold, the increase gradient threshold being either predefined or calculated based on the subset of the time series of readings, followed by a second period of decreasing CO2 concentration at a rate of change with respect to time exceeding a decrease gradient threshold, the decrease gradient threshold being either predefined or calculated based on the subset of the time series of readings, wherein if the first period and the second period are identified, and if a duration between the identified first and second periods is below a predefined threshold, the readings representing the first period and the second period are detected as a spike.

Optionally, a spike height threshold is applied to the first period of increasing CO2 concentration wherein a magnitude of increase in CO2 concentration represented by the first period is compared with the spike height threshold, and if the magnitude of increase does not meet the spike height threshold then the readings representing the first period and the second period are not detected as a spike.

Optionally, a spike in the CO2 concentration with respect to time, being a gastroparesis indicator spike or a gastric-duodenal transition indicator spike, is detected by identifying a local maximum feature being a singularity, discontinuity, or inflection point at more than a predefined threshold above a baseline value defined based on values preceding the feature.

Optionally, processing the readings to detect one or more gastroparesis indicator spikes, comprises: determining that the ingestible capsule device has been ingested by the subject and the ingestion timing.

Optionally, the ingestible capsule device houses an environmental temperature sensor to detect an environmental temperature at the ingestible capsule device, and determining that the ingestible capsule device has been ingested by the subject and the ingestion timing is by comparison of the environmental temperature represented by a signal from the environmental temperature sensor with a predefined temperature range for stomach of the subject or for the gastrointestinal tract of the subject.

Optionally, the ingestible capsule device houses a relative humidity sensor to detect relative humidity at the ingestible capsule device, and determining that the ingestible capsule device has been ingested by the subject and the ingestion timing is by comparison of the relative humidity represented by a signal from the environmental temperature sensor with a predefined relative humidity range for stomach of the subject or for the gastrointestinal tract of the subject; wherein determining that the ingestible capsule device has been ingested and the ingestion timing is based on one or both of the relative humidity and the environmental temperature being within the respective predefined range.

Optionally, the obtaining and processing steps, are performed by a processor housed within the ingestible capsule device.

Optionally, the diagnosing is performed by the processor housed within the ingestible capsule device.

Optionally, the ingestible capsule device comprises a wireless data transmitter, and the obtaining, processing, and diagnosing steps are performed at a processor of a receiver device external to the subject and configured to receive data from the wireless data transmitter, or at a remote processing apparatus in data communication with the receiver device.

Optionally, the diagnostic device comprises a wireless data transmitter, and the method further comprises preparing a report representing the detected one or more gastroparesis indicator spikes, and transmitting the report to a receiver device external to the subject via the wireless data transmitter, the diagnosing step being performed at the processor of the ingestible capsule device, at the receiver device or at a remote processing apparatus in data communication therewith.

Optionally, diagnosing gastroparesis or suspected gastroparesis includes calculating a score representing likelihood of gastroparesis being present in the subject, the likelihood score being calculated with reference to the detected one or more spikes relative to one or more reference cases.

Optionally, calculating the likelihood score is performed by a machine learning algorithm pre-trained with labelled training data, training data being representations of CO2 concentration measured by ingestible capsule devices during residence in stomachs of respective training subjects, each training subject being clinically diagnosed by a medical practitioner as being gastroparesis positive or gastroparesis negative, and the training data being labelled with the clinical diagnosis of the respective subject.

Embodiments may include an ingestible capsule device comprising: an ingestible indigestible bio-compatible housing; and, within the housing: a power source; sensor hardware including gas sensing apparatus; processor hardware; memory hardware; and a wireless data transmitter;
the memory hardware storing processing instructions which, when executed by the processor hardware, cause the processor hardware to perform a process comprising: obtaining data representing a time series of readings from the gas sensing apparatus, the time series of readings being taken during exposure of the gas sensing apparatus to a gas mixture at the ingestible capsule device during passage of the ingestible capsule device through a gastrointestinal tract of a subject, the subject having orally ingested the ingestible capsule device, each reading having a value, the values of the readings being sensitive to CO2 concentration in the gas mixture; processing the readings to detect one or more gastroparesis indicator spikes in the CO2 concentration with respect to time, a gastroparesis indicator spike being a spike in the CO2 concentration with respect to time at a timing after an ingestion timing of the ingestible capsule device and preceding a gastric-duodenal transition timing of the ingestible capsule device; based on the detected one or more gastroparesis indicator spikes in the CO2 concentration with respect to time, diagnosing gastroparesis or suspected gastroparesis.

Embodiments may include a system comprising an ingestible capsule device comprising an ingestible indigestible bio-compatible housing; and, within the housing: a power source; sensor hardware including gas sensing apparatus; processor hardware; memory hardware; and a wireless data transmitter; the system further comprising a receiver apparatus configured to obtain data representing a time series of readings from the gas sensing apparatus, the time series of readings being taken during exposure of the gas sensing apparatus to a gas mixture at the ingestible capsule device during passage of the ingestible capsule device through a gastrointestinal tract of a subject, the subject having orally ingested the ingestible capsule device, each reading having a value, the values of the readings being sensitive to CO2 concentration in the gas mixture; the receiver apparatus being further configured to, at the receiver apparatus or by causing processing to be performed at a remote processing apparatus in data communication with the receiver apparatus: process the readings to detect one or more gastroparesis indicator spikes in the CO2 concentration with respect to time, a gastroparesis indicator spike being a spike in the CO2 concentration with respect to time at a timing after an ingestion timing of the ingestible capsule device and preceding a gastric-duodenal transition timing of the ingestible capsule device; based on the detected one or more gastroparesis indicator spikes in the CO2 concentration with respect to time, diagnosing gastroparesis or suspected gastroparesis.

Embodiments may include a computer program which, when executed by a processor cooperating with a memory, causes the processor to perform a method of diagnosing gastroparesis or suspected gastroparesis, the method comprising: obtaining data representing a time series of readings from gas sensing apparatus housed within a ingestible capsule device orally ingested by a subjected, the time series of readings being taken during exposure of the gas sensing apparatus to a gas mixture at the ingestible capsule device during passage of the ingestible capsule device through a gastrointestinal tract of the subject, each reading having a value, the values of the readings being sensitive to CO2 concentration in the gas mixture; processing the readings to detect one or more gastroparesis indicator spikes in the CO2 concentration with respect to time, a gastroparesis indicator spike being a spike in the CO2 concentration with respect to time at a timing after an ingestion timing of the ingestible capsule device and preceding a gastric-duodenal transition timing of the ingestible capsule device; based on the detected one or more gastroparesis indicator spikes in the CO2 concentration with respect to time, diagnosing gastroparesis or suspected gastroparesis.

### DETAILED DESCRIPTION

Embodiments are described below, by way of example, with reference to the accompanying drawings, in which:

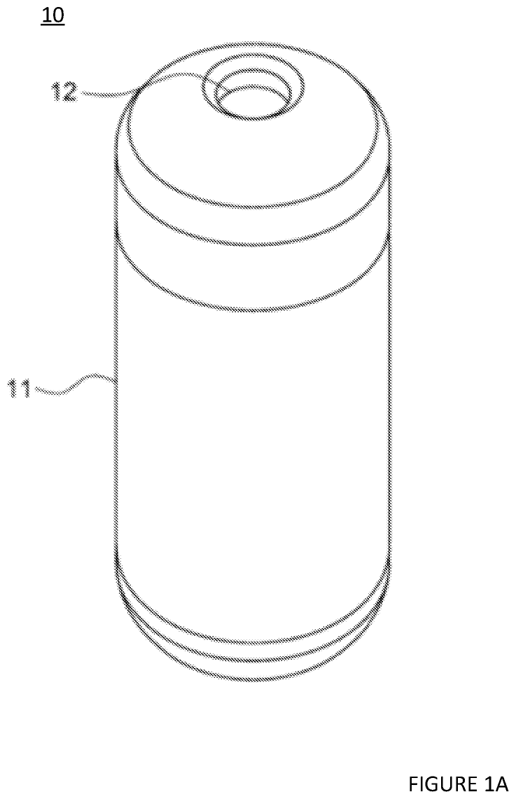

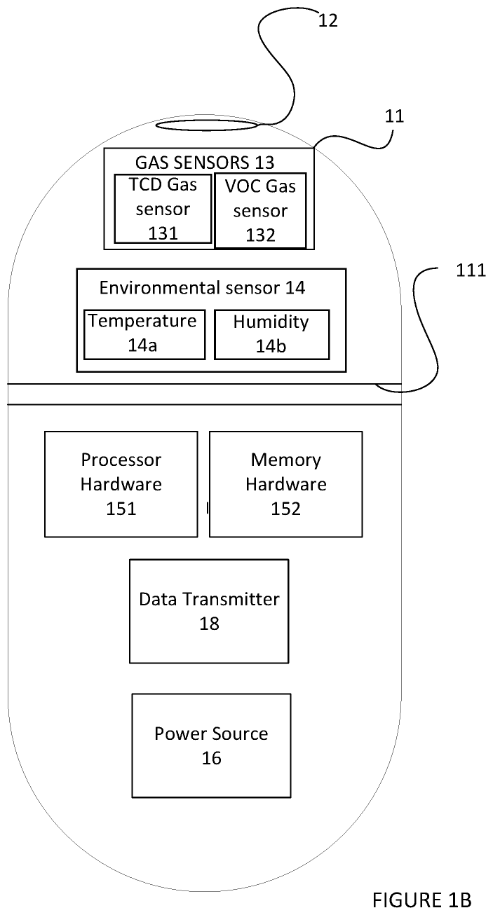

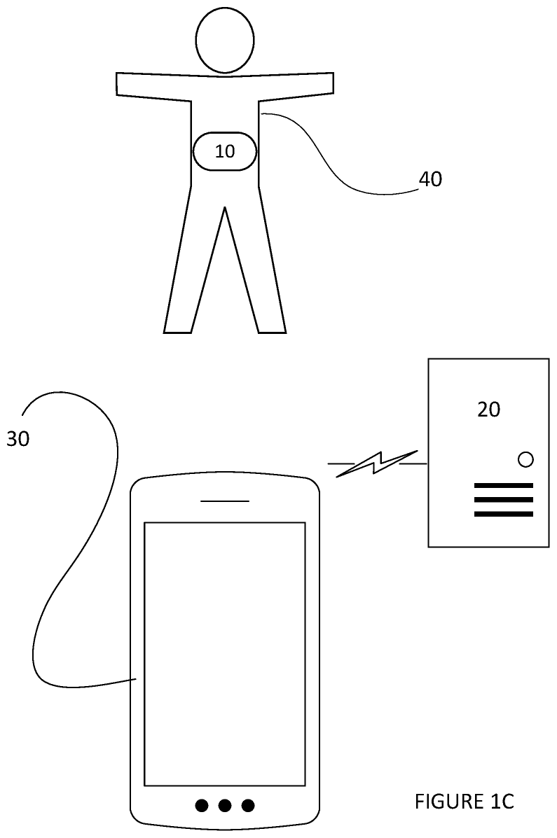

FIG. 1A illustrates an ingestible capsule device of an embodiment;

FIG. 1B illustrates an ingestible capsule device of an embodiment;

FIG. 1C illustrates a system encompassing an embodiment;

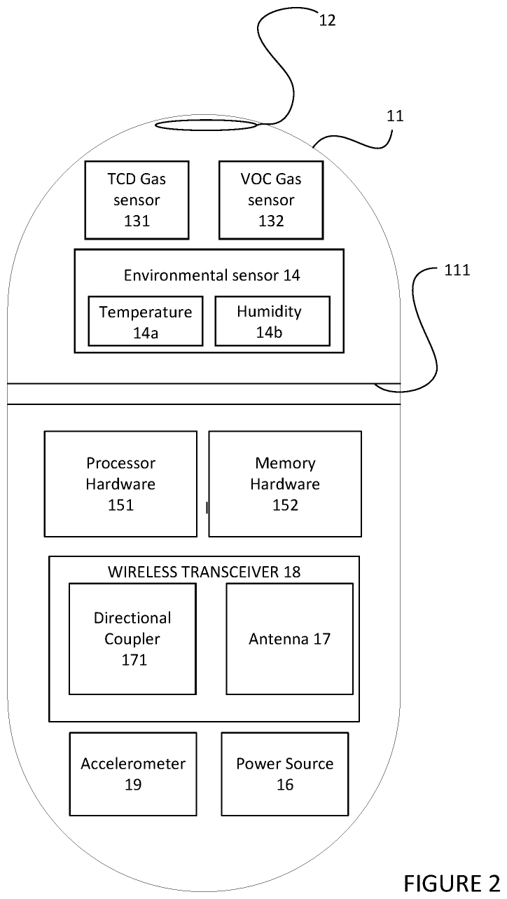

FIG. 2 illustrates a ingestible capsule device of an embodiment;

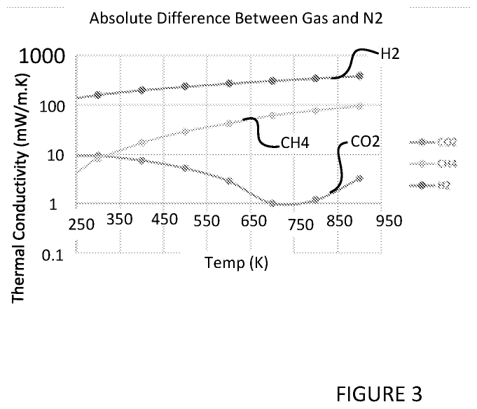

FIG. 3 illustrates changing sensitivity to constituent gases with operating temperature;

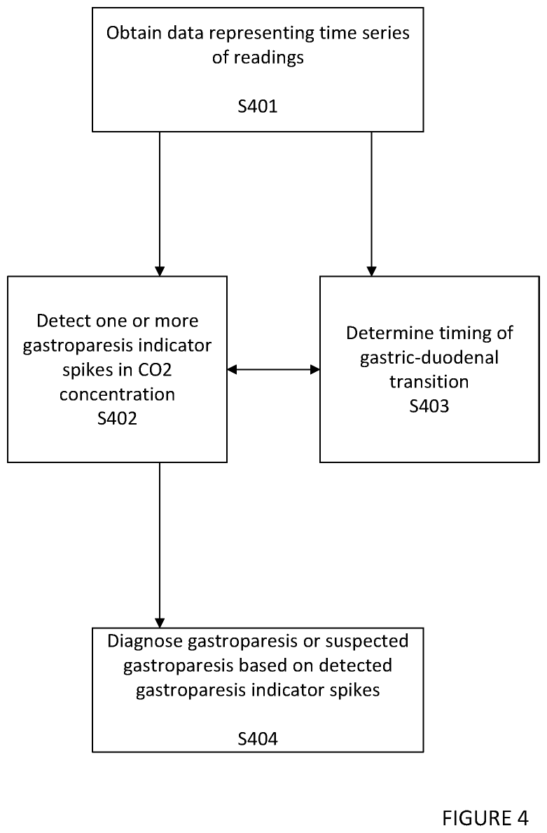

FIG. 4 illustrates a method according to an embodiment;

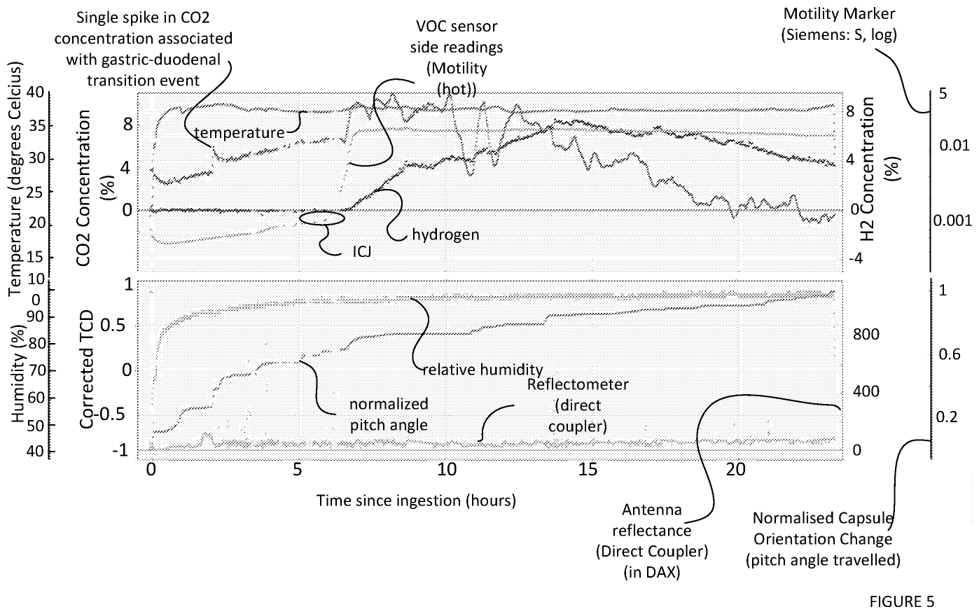

FIG. 5 illustrates sensor readings and indicators in a trial conducted on a healthy patient;

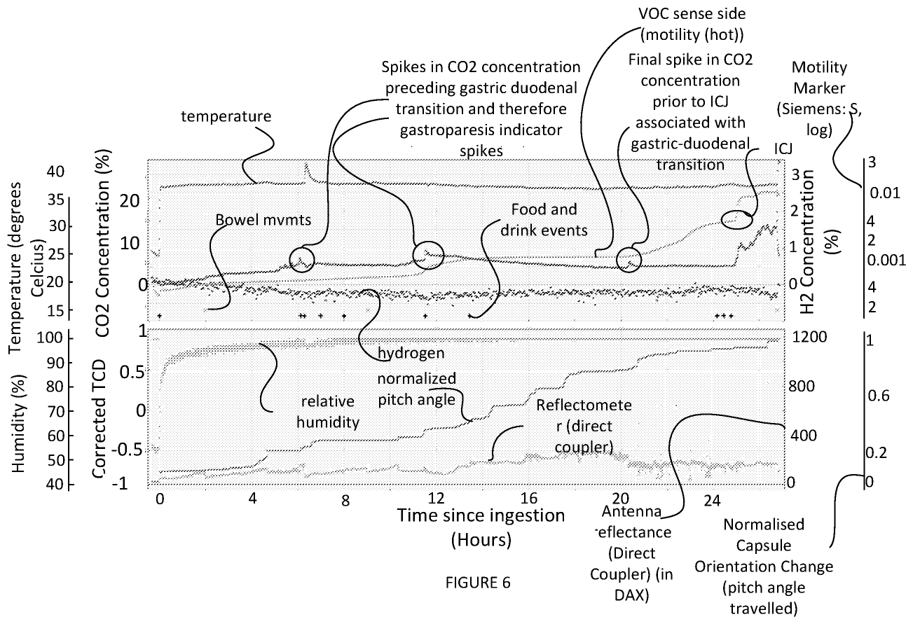

FIG. 6 illustrates sensor readings and indicators in a trial conducted on a gastroparesis positive patient;

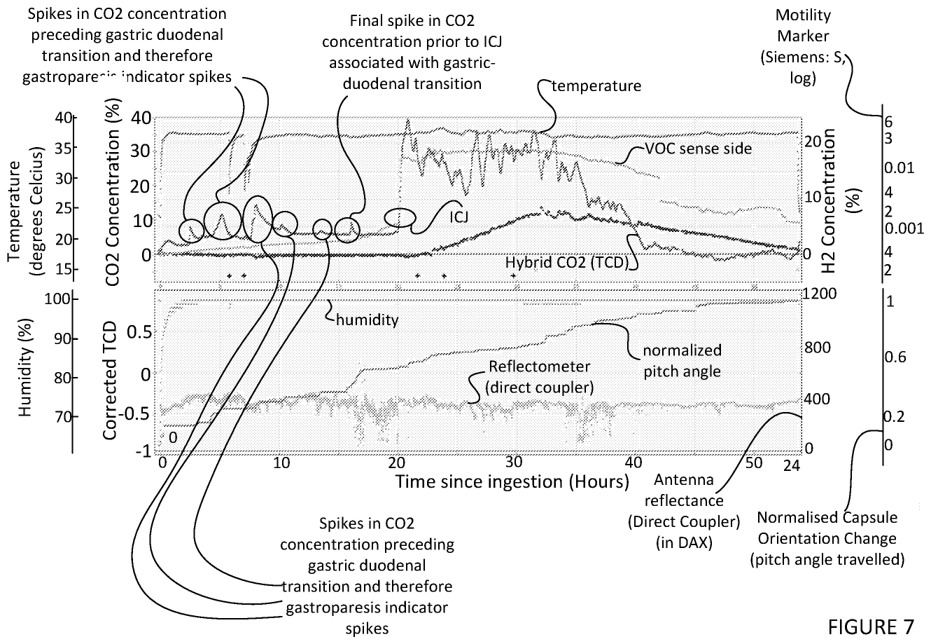

FIG. 7 illustrates sensor readings and indicators in a trial conducted on a gastroparesis positive patient;

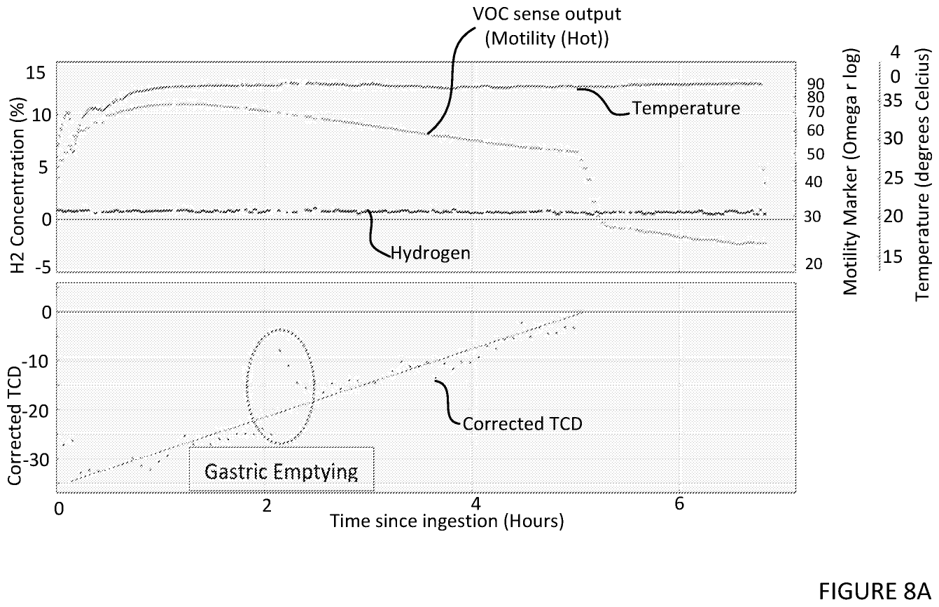

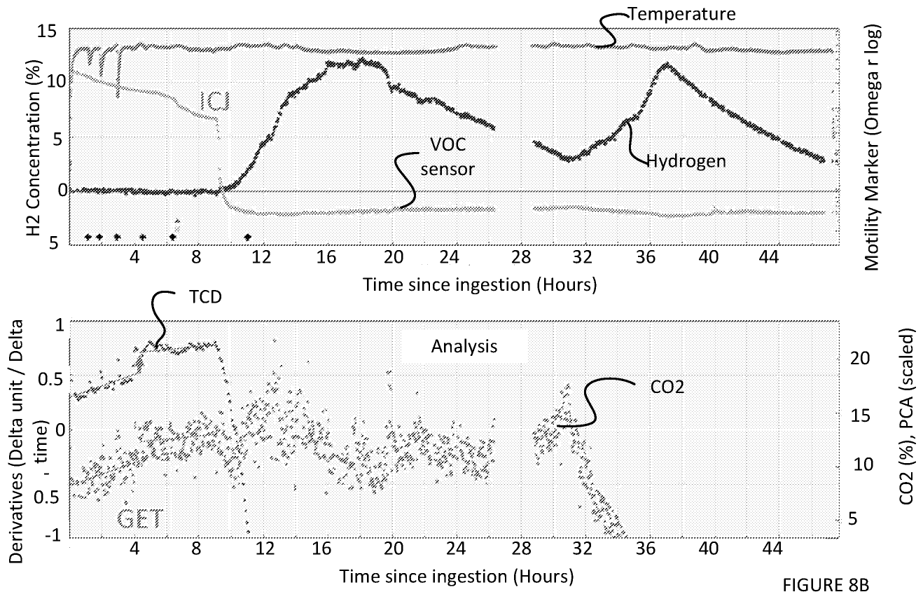

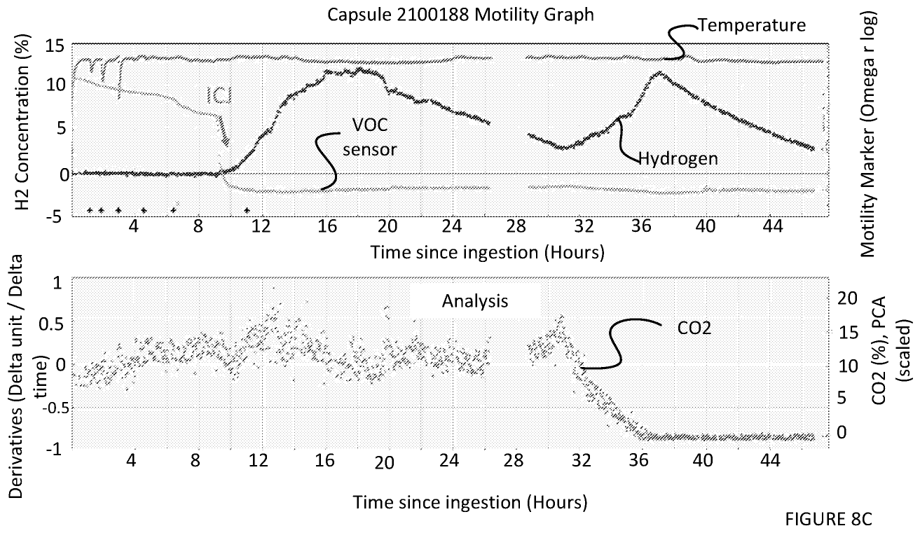

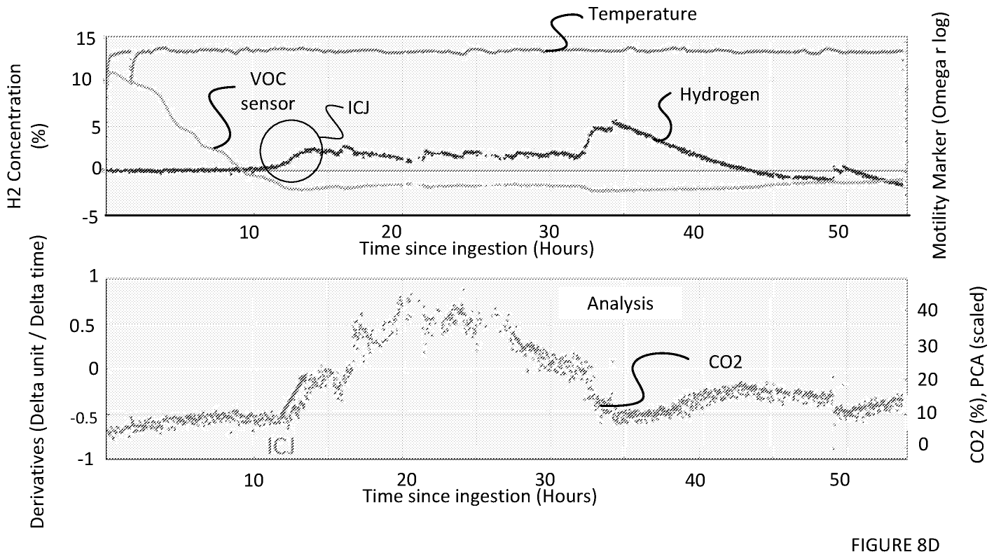

FIG. 8a illustrates a plot of data generated by an embodiment;

FIG. 8b illustrates a plot of data generated by an embodiment;

FIG. 8c illustrates a plot of data generated by an embodiment;

FIG. 8d illustrates a plot of data generated by an embodiment;

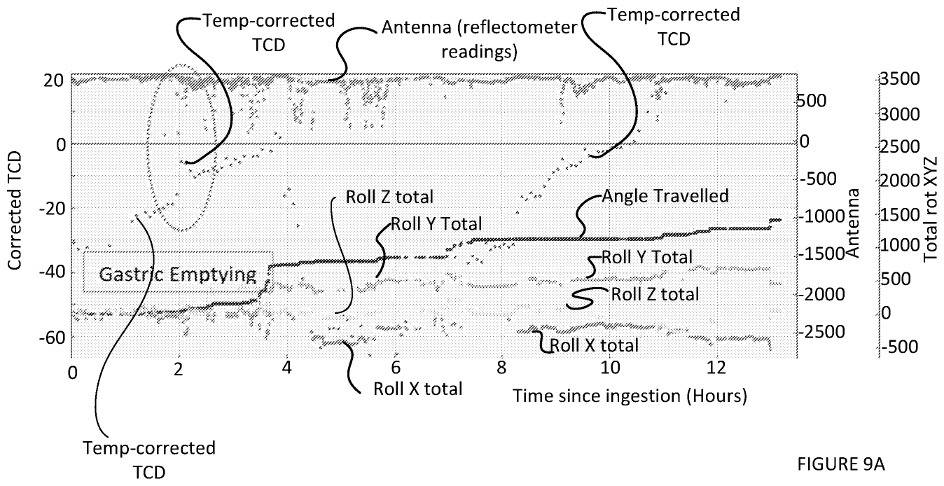

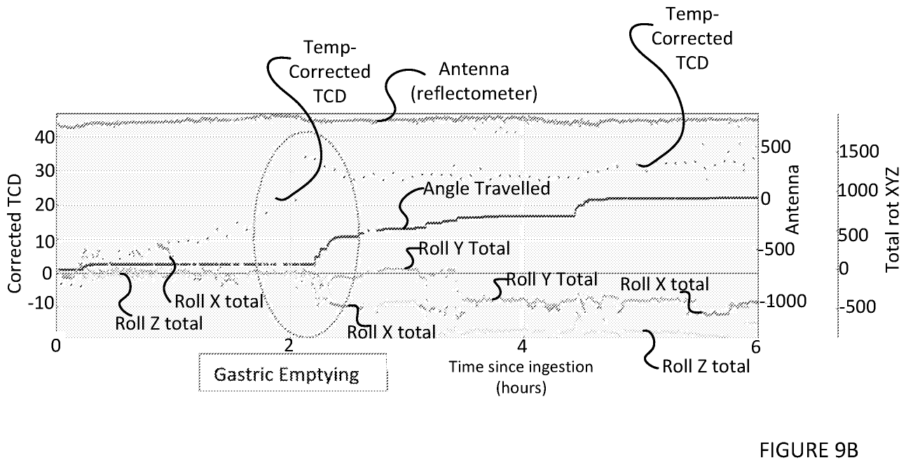

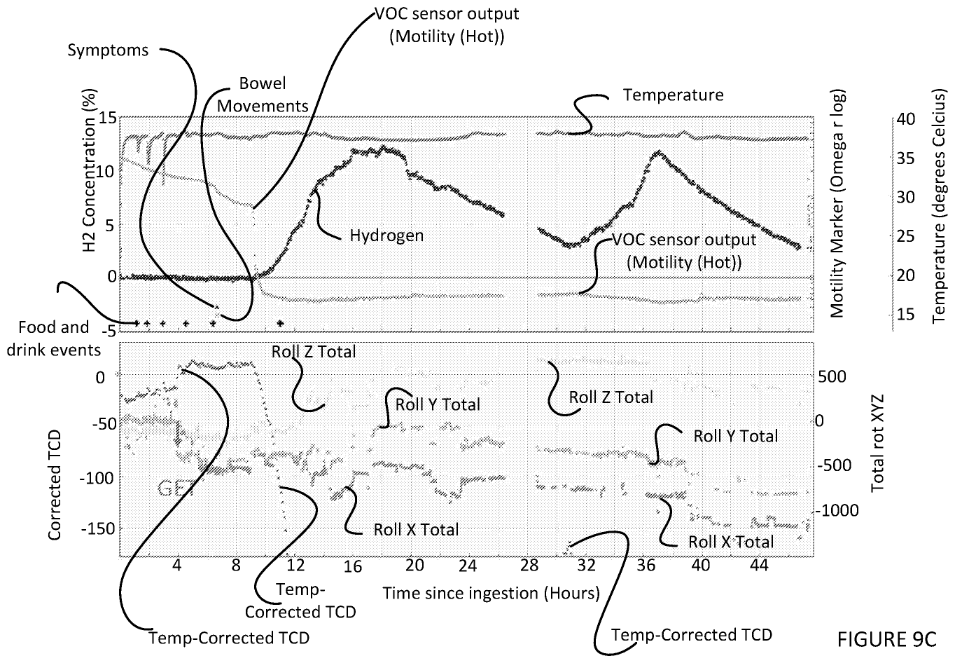

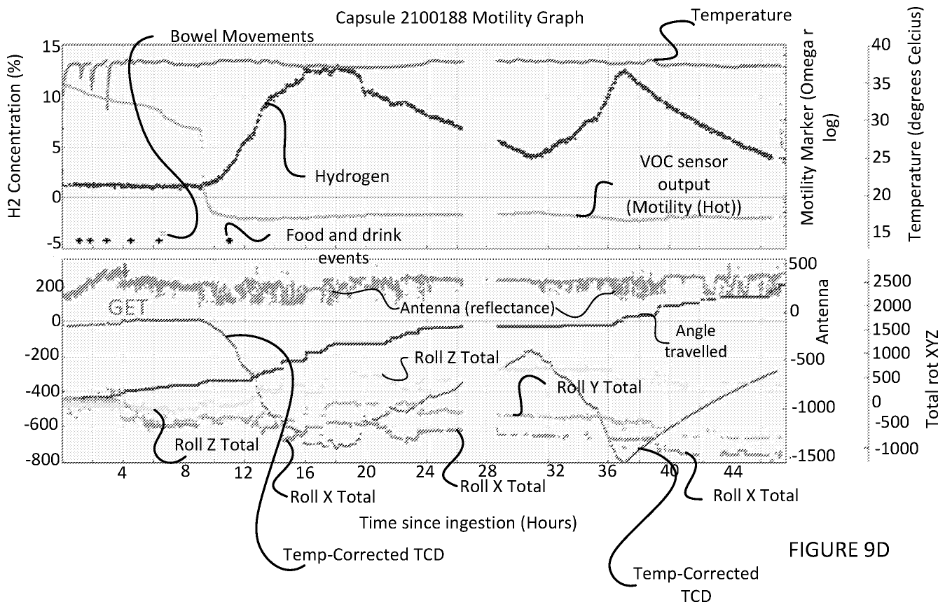

FIG. 9a illustrates a plot of data generated by an embodiment;

FIG. 9b illustrates a plot of data generated by an embodiment;

FIG. 9c illustrates a plot of data generated by an embodiment;

FIG. 9d illustrates a plot of data generated by an embodiment.

Methods generate detection of symptoms, diagnosis of a condition or a suspected condition by providing a ingestible capsule device 10 to a subject for ingestion. The ingestible capsule device 10 comprises one or more on-board sensors or quasi sensors which generate data in the form of signals or readings, which data when processed indicates the presence or otherwise of gastroparesis or suspected gastroparesis in the subject.

The term subject is used to refer to a patient and signifies subject of study or subject of diagnostic test. Gastroparesis is a condition for which a positive diagnosis may require verification by a clinical expert based on one or more other clinical tests and/or consultation with a patient. To that end, the term suspected gastroparesis is taken to be an indication that, based on the determination of the present method, gastroparesis is present in the subject (i.e. the motility of the stomach is slowed down versus a healthy subject), but that a clinical expert may use said indication one positive indicator among a combination of positive indicators, rather than a diagnosis per se. Noting that different clinicians may practice different procedures in this regard, owing to jurisdictional requirements or personal practice preferences.

FIGS. 1A to 2, with reference to FIG. 3, illustrate a ingestible capsule device 10 that may be utilised in embodiments.

### Ingestible Capsule Overview

FIGS. 1A and 1B illustrate an ingestible capsule 10. A system including the ingestible capsule 10 of FIGS. 1A and 1B is illustrated in FIG. 1C, during a live phase of the ingestible capsule 10 (i.e. while the ingestible capsule 10 is obtaining readings from within the GI tract of a subject mammal 40).

As shown in FIGS. 1A and 1B the typical capsule 10 consists of a housing such as a gas impermeable shell 11 which has an opening covered by a gas permeable membrane 12. A membrane 111 separates an exposed interior cavity exposed to the environmental gases entering the capsule 10 through the membrane 12 from a sealed-off interior cavity that is not exposed to the environmental gases.

As shown in FIG. 1C, the system, in addition to the capsule, further comprises a receiver apparatus 30 which receives data transmitted by the capsule from within the GI tract of the subject mammal during the live phase. Concurrently or subsequently, the receiver apparatus 30 processes the received data and may also upload some or all of the received data to a remote processing apparatus 20 such as a cloud-based service for further processing. The remote computer 20 may be a cloud resource, or may be a standalone computer at a clinician premise at which the subject is a patient, or may be a server (be it cloud-based or otherwise) at a service provider to which the clinician is a subscriber/customer/servicer user.

The remote processing apparatus 20 may be a server provided by or on behalf of a clinical centre at which subject 40 is a patient and taking responsibility for interpreting the results generated by the capsule 10 (i.e. the data transmission payload) and reporting them to the subject 40.

Connectivity between the capsule 10 and the receiver apparatus 30 is via the data transmitter on the capsule, which may be part of a wireless transceiver, for example a Bluetooth transceiver, which may operate according to a standard Bluetooth transmission protocol or according to Bluetooth Long Range (Coded-PHY) transmission protocol. Other operable communication technologies include LoRa, wifi and 433 MHz radio.

Internally the capsule 10 includes gas sensor hardware 131, 132, an environmental sensor 14, and processor hardware 151 and memory hardware 152. The processor hardware 151 and memory hardware 152 may be a microcontroller. The processor hardware 151 may be a microprocessor. The memory hardware 152 may be a non-volatile memory and the data stored thereon is accessible by the processor hardware 151. The processor hardware 151 processes data from signals received from the gas sensor hardware and the environmental sensor (and optionally also the reflectometer and accelerometer) and stores the processed data on the memory hardware 152. The processed data, or a portion thereof, is stored on the memory hardware 152 as a data transmission payload ready for transmission to a receiver apparatus 30 by the data transmitter 18.

By way of example, the capsule illustrated in FIG. 1C houses, as sensor hardware, an environmental sensor 14 in the form of a temperature sensor 14a and/or a humidity sensor 14b, gas sensors in the form of a TCD gas sensor 131 and a VOC gas sensor 132, an accelerometer 19, and a reflectometer. Embodiments may include any single or combination of those individual sensors. Alternatively or additionally, embodiments may include one or more sensors not illustrated in FIG. 1C such as a spectrophotometer, Surface Acoustic Wave sensor, and/or Bulk Acoustic Resonator Arrays.

The environmental sensor 14 may be a temperature sensor 14a or may be a temperature sensor 14a and a humidity sensor 14b. The gas sensors may be a TCD gas sensor 131, a VOC gas sensor 132, or a TCD gas sensor 131 and a VOC gas sensor 132. As illustrated in FIG. 2A, the internal electronics may also include a power source 16, for example, silver oxide batteries, an antenna 17, a wireless transceiver 18. The internal electronics may also include a reed switch. Other options for keeping the device switched off (or otherwise not consuming power) during storage include a physical switch pressed via a flexible part of the housing, or a photodetector and coupled field effect transistor that latches the microcontroller on when exposed to light. The internal electronics may further comprise an accelerometer 19 from which accelerometer data (i.e. a signal) is received at the processor hardware 151 for processing and subsequent storage at the memory hardware 152 and transmission by the data transmitter 18.

The gas sensors 131, 132 are less than several mm in dimension each and are sensitive to particular gas constituents including oxygen, hydrogen, carbon dioxide and methane. The VOC gas sensor 132 may be configured to give sensor side readings and driver or heater side readings. The heater side readings may be used to determine thermal conductivity of a surrounding gas and thereby the heater side readings of the VOC gas sensor are TCD readings. The sensor side readings are used to determine concentrations of volatile organic compounds in the surrounding gases and are VOC readings. The TCD gas sensor 131 may be, for example, a heating element coupled to a thermopile output, with the thermopile temperature, and therefore its output, varying due to energy conducted into the gas at the location of the capsule 10. The TCD gas sensor 131 measures rate of heat diffusion away from the heating element.

As illustrated in FIG. 3, the heater side of the VOC gas sensor 132 (operating as a TCD sensor) and the sensor side of the TCD gas sensor 131 have different operating ranges, so TCD readings from the two sensors collectively span a wider range of operating temperatures than either of the sensors individually. Both sensors have heating elements. The TCD gas sensor 131 has a low operating temperature but with a high precision. The heater side of the VOC gas sensor 132 increases the operating range but has a lower precision for TCD readings than the TCD sensor. The larger collective thermal range achieved by the two gas sensors 13 in concert enables better resolution of analytes in the event that the signals from the gas sensors are processed to resolve the analytes. The thermal conductivity of constituent gases in the gas mixture of the GI tract varies with temperature and so by obtaining TCD readings at different operating temperatures the different gases can be resolved from each other, by this technique, CO2 concentration readings are obtainable. This is leveraged in gas resolution processing, which is to determine identity and concentrations of constituent gases in the gas mixture surrounding the capsule 10, such as one or more from among CO2 and H2. The gas resolution processing may be performed on-board the gas capsule 10, at the receiver apparatus 30, or at a remote processing apparatus.

The gas sensors 13 are contained in a portion of the capsule 10 sealed from the power source 16 and other electronic components by a membrane 111. Such an arrangement minimises volume of the sensing headspace (i.e. the sealed portion) and minimises risk of a leak caused by a perforated membrane allowing GI-tract gases from the headspace to reach the power source. However, since the power source may be configured so that exposure to GI-tract gases does not adversely impact performance, the membrane may be omitted. That is, the membrane 111 is optional. The membrane 111 is permeable by electronic circuitry required to connect the components housed on either side. For example, wiring may pass through the membrane 111 in a sealed manner. The outer surface of the sealed portion of the capsule is composed of a selectively permeable membrane. Selectively permeable in the present context indicates that liquids are not allowed to permeate whereas gases are. The selectivity does not extend to allowing only a subset of gases to permeate. For example, the gas sensors 131 132 include respective heaters which are driven to heat sensing portions of the respective gas sensors 131 132 to temperatures at which sensor readings are obtained (i.e. a measurement temperature). The heaters may be driven in pulses so that there is temporal variation in the sensing portion temperature and so that measurement temperatures are obtained for periods sufficient to take readings but without consuming the power that would be required to sustain the measurement temperature continuously.

The gas sensors 13 are calibrated, so that a gas sensor reading can be used to identify the composition and concentration of a gas to which they are exposed. Calibration coefficients are gathered in manufacturing and testing and are applied to the recorded readings at the processing stage (i.e. by a server such as on the cloud or by an on-board processor 151). Otherwise, this calibration could be performed on the capsule 10, at the receiver apparatus 30, or on any device having access to the calibration coefficients and the recorded readings from the gas sensors 13. Such calibration relates to a gas resolution branch of processing concerned with measuring the concentration of constituent gases in the gas mixture at the capsule 10. Context for the outputs of that branch of processing is provided by a motility branch of processing, which determines (or predicts to within predefined confidence level) a location of the capsule 10 within the GI tract at which said gas mixture is found. In the motility (or location determination) processing branch, some calibration may also be required in seeking to find gastric-duodenal transition indicators, since ingested foodstuffs at different temperatures change the environmental temperature in the stomach, which influences rate of heat diffusion. In the case of gas sensor readings taken after ingestion and before the gastric-duodenal transition (i.e. whilst the capsule 10 is in the stomach), processing of readings may include applying a moderation to TCD readings, from either gas sensor, in order to correct for variations in environmental temperature, based on environmental temperature readings by the temperature sensor 14a. TCD readings are effectively measuring rate of heat loss to surroundings, and so accuracy is improved by measuring the temperature of the surroundings rather than by relying on assumption (i.e. prior knowledge of internal temperature of the subject mammal). However, the processing may rely on assumption, for example, if there is some issue with the temperature sensor readings, or, for example, if the level of accuracy provided by assumption is acceptable in a particular implementation. Gastric temperature may vary based on, for example, ingestion of liquids or foodstuffs by the subject mammal, or physical activity undertaken by the subject mammal 40. Environmental temperature is a term used in this document to refer to the temperature of the environment in which the capsule 10 is located, as distinct from operational temperatures of the gas sensors 13.The sensitivity of the gas sensors 13 to different constituent gases vary according to the operating temperature of the sensors and the processing of the readings includes calibrating (also referred to as moderating or correcting) readings from the gas sensors according to contemporaneous operating temperature and optionally also according to contemporaneous environmental temperature.

It is noted that the motility branch of processing and the gas resolution branch of processing are not independent of one another. Some motility indicators (i.e. features or characteristics of sensor output signals used to determine timing of motility events) may be found in readings of concentration of a single analyte gas in the gas mixture at the capsule, obtained by processing the output of one or more of the gas sensors 13.

In addition to the gas sensors 13 and the temperature sensor 14a, the capsule electronics further include processor hardware 151, memory hardware 152, a power source 16, an antenna 17, a wireless transmitter 18, and optionally a reed switch. The wireless transmitter 18 operates in concert with the antenna 17 to transmit readings from the sensors (collectively referring to the gas sensors 13 and the temperature sensor 14a, and optionally also the accelerometer 19 and reflectometer) to a receiver apparatus 30 for processing thereon or at a remote processing apparatus to which the receiver apparatus is in data communication, or the processor hardware 151 processes the signals received from the sensors to identify motility indicators (or otherwise to extract information from the sensor readings).

The wireless transmitter 18 (also referred to as data transmitter 18) may be provided as part of a wireless transceiver 18. The wireless transceiver 18 includes an antenna 17. Optionally, the wireless transceiver 18 also includes a directional coupler 171. The wireless transceiver 18 may transmit data in accordance with the Bluetooth protocol, the Bluetooth Long Range (Coded-PHY) protocol, the LoRa protocol, the wifi protocol, or using another mode of transmission such as 433 MHz radio wave transmission.

FIG. 2A illustrates the antenna 17 and directional coupler 171 as elements of the wireless transmitter 18, since the antenna is the physical means by which the wireless transmitter 18 transmits data to the receiver apparatus 30. The wireless transmitter 18 is also configured to buffer data for transmission. The wireless transmitter 18 may also be configured to encode the data with a code unique to the capsule 10 among a population of like capsules 10.

Interconnections between electronic components in FIG. 3 may be via a central bus. This is one example of how power and data may be distributed between components. Other circuitry architecture may be implemented, for example, all connections may be via a microcontroller which coordinates distribution of data and power between components. The sensors (the TCD sensor 131, the VOC sensor 132, the temperature sensor 14a, the accelerometer 19, and the directional coupler 171) take readings under the instruction of a microcontroller, powered by the power source 16, and transfer the readings (or results of processing the readings) to the wireless transmitter 18 for transmission to the receiver apparatus 30 via the antenna 17. For example, the processor hardware 151 and memory hardware 152 may collectively be referred to as a microcontroller.

The dimension of the capsule may be less than 11.2 mm in diameter and less than 27.8 mm in length. The housing of the capsule 10 may be made of indigestible polymer, which is biocompatible. The housing may be smooth and non-sticky to allow its passage in the shortest possible time and to minimise risk of any capsule retention. Optionally, the ingestible capsule may be less than 32.3 mm in length and less than 11.6 mm in diameter.

The antenna 17 may be in series with a directional coupler 171. The directional coupler 171 and the antenna 17 are configured as a reflectometer. The reflectometer measures the amplitude of reflected signals by means of a diode detector. The measurements of the reflectometer are readings that represent electromagnetic properties of material in the vicinity of the capsule. The reflectometer readings provide a basis for differentiating between gaseous, liquid, and solid matter at the location of the capsule in the GI tract. Readings of the reflectometer enable the antenna 17 and directional coupler 171 to operate in cooperation as an environmental dielectric sensor.

The readings of the ingestible capsule 10, which include one or more from among readings from: the temperature sensor 14a, the heater side 132b of the VOC gas sensor 132, the sensor side 132a of the VOC gas sensor 132, and the TCD gas sensor 131, may also include readings of the reflectometer. Hence, change in capsule location within the GI tract causes a change in reflectometer readings, and therefore provide an indicator that a transition event between two sections of the GI tract has occurred. The ingestible capsule 10 may further comprise an accelerometer 19. The accelerometer 19 may be a tri-axial accelerometer. A rate of change of angular position or orientation of the capsule 10 is somewhat dependent upon location within the GI tract, and therefore accelerometer readings provide an indicator that a transition event between two sections of the GI tract has occurred. The accelerometer readings may measure angular acceleration about three axes of rotation, wherein the three axes of rotation may be mutually orthogonal.

### Processor Hardware, Memory Hardware

The processor hardware and memory hardware may be separate components or may be part of the same single integrated chip. The processor hardware and memory hardware are selected according to the particular implementation requirements of each design or version of the capsule 10, noting that constraints such as power consumption, cost, data throughput, size of data transmission payload, etc, will vary between designs or versions. The processor hardware may be a processor or a plurality of interconnected processors.

### Pairing

The wireless data transmitter may be a Bluetooth transmitter, a wifi transmitter, a radio transmitter, or another form of wireless data transmitter. A radio transmitter may be configured to transmit in the 433 MHz band. In any case, the wireless data transmitter may be provided as part of a wireless data transceiver. For example, the wireless data transceiver may receive signals at least in performing pairing or any other form of coupling to a recipient device 30. The capsule 10 may be configured to enter into a wireless pairing or coupling mode immediately upon initiation (i.e. first power-on), wherein a subject or another user is instructed (via written instructions or via an application running on the recipient device itself) to pair or couple the capsule 10 to the recipient device 30 prior to ingestion of the capsule 10. However, embodiments may be configured such that pairing or coupling is not necessary, for example the capsule 10 may be configured to broadcast data to a recipient device in a data transmission technique that is agnostic to pairing or coupling status, as discussed in more detail below.

### Data Transmission Techniques

There are two principal data transmission techniques, which ingestible capsules may be configured to use either or both of, depending on implementation details (i.e. use case). In a post-excretion data transmission technique, signals from the sensors are received at the processor hardware 151 (utilising also the storage capabilities of the memory hardware 152) and processed on-board the capsule 10 in order to identify and record motility indicators (and optionally also other characteristics of the sensor output or sensor readings of interest or groups of sensor readings of interest) and the recorded motility indicators (and optionally also the other characteristics, metrics, and readings or groups of readings of interest, such as peak H2, area under a plot of H2 against time, number of gastroparesis indicator spikes, height of gastroparesis indicator spikes, aggregate gastroparesis indicator spike height, aggregate area under gastroparesis indicator spikes) are stored on the memory hardware 152 as a data transmission payload. Other characteristics and readings or groups of readings of interest may include, for example, maximum or minimum readings from specific sensors or from metrics calculated by combining sensors. The maximum or minimum readings may be local maximum or local minimum readings, wherein local is defined by, for example, predefined timings or motility events determined to have occurred by the capsule 10 itself. A specific example is maximum or minimum H2 concentration, which is a metric calculated from the gas sensor readings by an appropriately calibrated processor hardware. The data transmission payload is transmitted by the wireless transceiver once excretion of the capsule 10 from the GI tract is detected (for example by the temperature sensor 14a signal and/or by the accelerometer 19 signal). Metrics further include peak H2 level or value, timing of peak H2, and total H2 (area under the curve). Such metrics may be calculated by the on-board processor hardware 151 during passage through the GI tract of the subject, and transmitted away from the capsule 10 to a receiver device in post-excretion transmission as part of a report or otherwise.

In the post-excretion data transmission technique, the transmission may be via a Bluetooth transmission mode that is not dependent upon pairing status. That is, for example, if the Bluetooth transceiver is paired to a receiver device then it transmits the data transmission payload to the paired receiver device, and if the Bluetooth transceiver is unpaired then it broadcasts the data transmission payload to a recipient device in the absence of pairing in an inquiry mode (which may be referred to as discovery mode or beacon mode). Bluetooth protocol has an inquiry mode in which a device broadcasts a unique identifier, name and other information. The data transmission payload, or part thereof, may comprise or be included in the said other information. In particular, the data transmission payload may be prioritised or otherwise filtered by the processor hardware 151 so that information deemed particular important such as an indication that excretion has occurred (it is important for clinical reasons to know that the capsule 10 has been excreted) and potentially information such as timing of determined motility events, is transferred away from the capsule 10 in preference to other information. Following the inquiry mode transmission, the transceiver may again attempt to pair, connect, or otherwise couple, with the recipient device, and if successful, to transmit the remainder of the data transmission payload. Of course, said pairing, connecting, or coupling, may have been performed initially pre-ingestion so that post-excretion the Bluetooth transceiver is attempting to re-pair, re-connect, or re-couple, with the receiver device 30. It is noted that the present discussion uses Bluetooth as an example of a transmission protocol, but that the same techniques could be applied to different transmission protocols.

In the event that there is data transmission payload pending transmission away from the capsule 10 after the broadcast of the unique identifier, name, and other information during the Bluetooth inquiry mode, then capsule 10 may be configured to initiate or re-initiate a data communication connection (i.e. a pairing or re-pairing) with a receiver device 30. Upon successful initiation or re-initiation of the communication connection, transmission of the said data transmission payload pending transmission away from the capsule 10 is performed whilst the data communication connection remains active.

The Bluetooth transceiver 18, or any other wireless data transmitter 18, may be configured to automatically re-connect following an initial (i.e. pre-ingestion) connection to a receiver device 30. The receiver device 30 may run an app or web app to guide the subject in terms of how to ingest the capsule 10, to notify the subject that the excretion event has been determined, and optionally also that the data transmission payload has been successfully transmitted to the receiver device 30 and so the capsule 10 may be flushed away. It is noted that the terms pair, connect, and couple, are interchangeable in the present document, each representing the establishment of a wireless connection between two devices for wireless data transfer.

It is noted that data transmission payload may be being transmitted throughout passage of the capsule 10 through the GI tract, dependent upon pairing, coupling, or connection to the receiver device 30. However, confirmation that occurrence of an excretion event has been determined by the capsule is information that is of particular importance since safety of capsule 10 is reliant on the capsule 10 being excreted. Therefore, information representing determination of occurrence of the excretion event (i.e. a report thereof) is prioritised and may be transmitted in a broadcast or inquiry mode, whereas the remaining data transmission payload is transmitted once connection between the wireless data transmitter 18 and the receiver device 30 is established.

In Bluetooth inquiry mode, data can be transmitted to the receiver apparatus 30, or to any Bluetooth receiver apparatus within range of the capsule 10, without pairing. The wireless transceiver 18 is operable in a Bluetooth inquiry mode or a Bluetooth long range (Coded-PHY) mode. Capsules 10 may store and transmit among the data transmission payload readings from one or more sensors representing a predefined period either side of the identified motility indicators. For example, gas sensor signals only, or for all sensors. Such readings may be used to add confidence to the identified motility indicators in terms of determining whether or not a motility event has occurred, and/or may provide other information useful in a health or clinical context.

More generally, data transmitted according to the post-excretion data transmission technique may be any of the data transmission payload that has not already been transmitted. For example, the wireless data transmitter 18 may be configured to transmit the data transmission payload to a paired receiver apparatus while still in the GI tract (this transmission is referred to herein as pre-excretion data transmission technique). However, owing to issues such as signal attenuation, noise, power supply issues, temporary pairing failure, or if pairing was never performed in the first place, or for any other reason, some or all of the data transmission payload may be pending transmission at the point of excretion. In that case, the remaining data transmission payload is transmitted according to the post-excretion data transmission technique once excretion is detected. It is noted that down-sampling of the data transmission payload may be performed prior to transmission via the post-excretion data transmission technique. Furthermore it is noted that some elements of the data transmission payload may be prevented from transmission via the post-excretion data transmission technique. For example, since bandwidth, and also time within which to transmit, may be limited, it may be that the motility event indicators and diagnostic indicators themselves are included, but that sensor readings are excluded from the data to be transmitted according to the post-excretion data transmission technique.

In a pre-excretion data transmission technique, the sensor signals are transmitted continuously by the wireless transceiver 18. In the pre-excretion data transmission technique, the process hardware 151 coordinates the receipt of the signals from the sensors and the storage at the memory hardware 152 for transmission by the wireless transceiver 18.

In the example of a Bluetooth wireless transceiver 18, in the pre-excretion transmission technique the transceiver may be operated according to a long-range or Coded PHY Bluetooth transmission procedure, such as BTLE Coded PHY. A signal power enhancement of around 10 dB is achievable via BTLE Coded PHY Bluetooth transmission procedure.

During a data transmission phase of the ingestible capsule 10 (i.e. which in the post-excretion data transmission technique is in a short burst post-excretion and in the pre-excretion data transmission technique is continuous while the ingestible capsule 10 is in use, that is, in the GI tract of a subject mammal 40 and obtaining and transmitting readings) the wireless transmitter 18 transmits the readings to a receiver apparatus 30, which may be a dedicated device for receiving and storing the readings (and optionally with a user interface) or may be a multi-function device such as a mobile phone (such as a smart phone). The mobile phone may be running an application which processes some or all of the data transmission payload to generate a motility report or diagnosis of a medical condition based on motility indicators and diagnostic indicators either included in the data transmission payload or derivable therefrom. Alternatively, the application may be configured to transmit the data transmission payload on to a server or another processing apparatus to generate the motility report or diagnosis based on the data transmission payload. The subject mammal need not remain within a specific range of the remote computer 20 during the live phase. Capsules 10 equipped with a Bluetooth transceiver 18 may communicate directly with a smartphone of a user, which obviates any need for a dedicated receiver apparatus (the smartphone taking on the role of receiver apparatus 30). The receiver apparatus 30 (whether a dedicated device or a mobile phone or tablet computer) may process the readings itself or may upload the readings to a remote computer 20 for processing (i.e. identifying motility indicators, determining motility event timings, resolving gas analytes). The upload may be continuous during a live phase of the capsule, or the upload may be after the live phase of the capsule is terminated. The receiver apparatus 30 may also store the readings, so that loss of connectivity between the receiver apparatus 30 and a remote processing apparatus is not critical.

The on-board processor 151 may apply one or more processing or pre-processing steps, as discussed in more detail below. Digitisation of the readings is performed either by the sensors themselves, by the processor 151 or by the wireless transceiver 18. The digitised readings are transmitted via the antenna 17. The readings of the capsule 10 are made at an instant in time and are associated with the instant in time at which they are made. For example, a time stamp may be associated with the readings by the microcontroller 15, the wireless transmitter 18, or at the receiver apparatus 30 or remote computer 20. For example, if readings are made and transmitted more-or-less instantaneously (i.e. within one second or a few seconds) by the wireless transmitter 18 then the time of receipt by the receiver apparatus may be associated with the readings as a time stamp. Processing of the readings discussed further below is somewhat dependent on the relative timings of the readings (i.e. so that contemporaneous readings from the different sensors can be identified as contemporaneous), however accuracy to the level of one second, a few seconds, or 10 seconds, is sufficient.

In a hybrid mode, capsules 10 may combine the two data transmission techniques. For example, the capsule 10 may process sensor readings on-board to identify motility markers (and optionally also other readings or groups of readings of interest) for transmission in Bluetooth inquiry mode immediately post-excretion. In addition, the capsule 10 may continuously transmit sensor readings to a paired receiver apparatus. Optionally, the continuous transmission may be of the gas sensor signals only, or gas sensor signals and temperature sensor signals required to calibrate gas sensor signals. Gas sensor signals are of particular interest in providing health and clinical information, particularly once combined with motility indicators provided by the other sensors such as accelerometer, reflectometer. Gas sensor signals may be downsampled or subject to other compression techniques by the on-board processor prior to transmission. Optionally, the on-board processor hardware 151 may apply one or more filters, such as a high pass or low pass filter applied to the values themselves or to the derivative with respect to time, so that only gas sensor signals meeting particular thresholds are included in the data transmission payload. Metrics representing gas sensor signals, such as peak of a derived H2 value, aggregate area under gastroparesis indicator spikes in CO2 concentration plot, or area under a plot of derived H2 value with respect to time, may be maintained and transmitted away from the capsule 10.

For capsules 10 configured to perform data transmission during passage through the GI tract (i.e. pre-excretion data transmission technique), commercial bands (such as 433 MHz) are used by the antenna 17 as electromagnetic waves in this frequency range can safely penetrate the mammalian tissues 40. Bluetooth may also be used in such capsules, wherein Bluetooth may be long-range Bluetooth (coded-PHY), particularly when BMI of the subject (human) is above a threshold, or a high level of attenuation is expected for some other reason. Other commercial bands and protocols may be used in various applications, such as LoRa. Coding may be applied at the digitisation stage to assure that the data transmitted by the capsule 10 is distinguishable from data transmitted by other similar capsules 10. The transmission antenna 17 may be, for example, a pseudo patch type for transmitting data to the outside of the body data acquisition system.

Power source 16 is a battery or super capacitor that can supply the power for the sensors and electronic circuits including the processor hardware 151 and memory hardware 152. A life time of at least 48 hours may be set as a minimum requirement for digestive tract capsules. A number of silver oxide batteries in the power source 16 is configurable, depending on the needed life time and other specifications for the capsule. For example, long-range Bluetooth may consume more power than standard Bluetooth. Capsules may be configured to switch from long-range Bluetooth transmission to standard Bluetooth transmission once the stored energy in the battery (or batteries) drops below a predefined threshold, wherein the on-board processor is configured to monitor stored energy level.

### Data Processing Approaches

The on-board sensors generate a large amount of data. Limitations such as energy capacity of power source mean that it may be preferable to process some data on-board the capsule 10 in order to extract a (relatively smaller) data transmission payload from the (relatively larger) generated data. In addition to extraction, data processing techniques may summarise or otherwise represent the generated data in order to reduce the size of the data transmission payload. The processor hardware 151 may be configured to prioritise contents of the data transmission payload. In particular, data representing that the excretion event has been determined and the timing thereof may be given highest priority (i.e. transmitted in preference to other content of the data transmission payload pending transmission at the same time as the data representing that the excretion event is pending transmission). Processing outcome such as positive or negative diagnosis, optionally along with an indicator of severity, of gastroparesis may be included in the prioritised contents.

It will be appreciated that there is a full spectrum of possibilities between, at one extreme, transmitting all generated data away from the capsule 10 for processing elsewhere (i.e. from capsule perspective a high data transmission burden and low data processing burden) and at the other extreme performing a high degree of processing on board to determine results including timings of motility events to a high degree of certainty and even to diagnose specific health conditions or ailments, and only transmitting the said processing results (i.e. from capsule perspective a low data transmission burden and high data processing burden).

Embodiments are configurable at the design stage according to implementation requirements to combine data processing and data transmission in a manner that enables data processing to occur, whether on-board or at a receiving apparatus 30 or remote data processing apparatus 20, to determine motility events, and other gut health indicators such as gas constituent concentrations at one or more locations/timings in the GI tract, and to identify or detect diagnostic indicators.

It is noted that the data transmission techniques detailed above may be considered orthogonal to the data processing approaches, in the sense that which data transmission technique, or combination of data transmission techniques, is selected does not necessarily dictate the data processing approach. However, of course, the data transmission capacity of each technique must be considered in deciding how much processing to perform on-board the capsule 10, noting that, in general, processing on-board the capsule 10 reduces the size of the data transmission payload, on the assumption that processing results are included in the data transmission payload in place of readings processed to generate said processing results.

The term signal may refer to the output signal produced by a sensor, whereas the term reading may refer to a specific measurement of the signal taken at or otherwise associated with an instant in time, which instant in time may be included with or associated with the reading explicitly or implicitly (i.e. if the reading is the 1000^{th} reading in a series and readings are taken at a rate of 1 Hz and the timing of the first reading in the series is known, then the position of the reading in the series implicitly represents the timing). Time stamps or other timing indicators may be provided by the processor hardware 151. Value is used to refer specifically to the value of signal contained in a reading, noting that a reading may also include metadata such as a time stamp. Nonetheless, it is evident that each reading has a value and that therefore where two readings are compared with one another, it is specifically the values that are compared.

On-board processing may be performed in more-or-less real time, allowing for latency caused by transfer between components and processing itself. Alternatively, the readings may be received by a receiver apparatus 30 processed thereby and/or stored for upload and processing retrospectively by a remote processing apparatus 20. Dependencies may exist between indicators or markers in the data which constrain an order in which readings are processed.

Since the diagnostic method specifically relates to analysing data representing an increase in CO2 concentration in the gas mixture at the capsule 10, an on-board processor 151 may extract readings representing a spike in CO2 concentration from those not representing an increase, and add the extracted readings to the data transmission payload whilst discarding the remainder. For example, by maintaining an average such as a rolling average as a baseline, and determining a spike or spike candidate by one or a predefined number of readings in a row being above the baseline or above the baseline by more than a threshold, which threshold may be predefined or may be determined on the fly, such as a predefined proportion of the baseline, or a predefined number of standard deviations (the spike or candidate spike ending when readings return to the baseline or cease to exceed the threshold). So that the on-board processor 151 performs pre-processing, and an off-board processor receives the extracted readings to perform the diagnostic method. Rather than transmission to an off-board processor for diagnostic processing, the extracted readings may be stored on memory hardware on-board the capsule for on-board processing. For example, extracted readings may be stored to be processed once it has been determined that the capsule 10 has exited the stomach, i.e. that the capsule 10 has undergone gastric-duodenal transition.

Gastric-duodenal transition of the capsule 10 is also associated with a spike in CO2 concentration, so spikes may be identified or detected generically and then distinguished as either a gastroparesis indicator spike or a gastric-duodenal transition indicator spike based on chronology, wherein a latest spike is determined to be associated with gastric-duodenal transition of the capsule 10, and preceding spikes are determined to be gastroparesis indicator spikes. Optionally, the gastric-duodenal transition timing may be determined from, for example, H2 concentration readings, accelerometer readings, and/or reflectometer readings, so that based on that timing the CO2 concentration spike at gastric-duodenal transition is not mis-identified as a gastroparesis indicator spike, because it does not precede gastric-duodenal transition. Embodiments may incorporate a five-, ten-, or fifteen minute buffer into the determined gastric-duodenal transition timing, so that any CO2 concentration spike within the buffer preceding the determined gastric-duodenal transition timing is not detected as a gastroparesis indicator spike.

The on-board processor 151 may be configured to perform the diagnostic method by executing processing instructions stored on the on-board memory hardware 152. Data processing overhead is increased in this case, which increases performance requirements and thus cost of the on-board processor 151 and memory 152, but reduces the data transmission overhead thus suppressing performance requirements of the wireless data transmitter 18. On the assumption that processing data on-board consumes less energy than transmitting said data to a receiver apparatus 30 for off-board processing, the on-board processing case also reduces stored energy requirements at the power source 16.

In the off-board processing case, the processing may be executed at a receiver apparatus 30 in direct communication with the ingestible capsule device 10, or at a computing apparatus 20 remote from the receiver apparatus 30 but in data communication therewith. For example, the receiver apparatus 30 may be a dedicated device configured to receive signals transmitted by the wireless data transmitter 18, such as signals transmitted in the 433 MHz radio band. Alternatively the receiver apparatus 30 may be a general purpose computing apparatus such as a smartphone or tablet computer configured to receive signals transmitted by the wireless data transmitter 18, such as signals transmitted according to the Bluetooth transmission protocol or according to the LoRa transmission protocol.

Communication between the capsule 10 and the receiver device 30 may be via a wireless data transmitter 18 on the capsule 10 configured to transmit signals according to the LoRa data transmission protocol.

Communication between the capsule 10 and the receiver device 30 may be via a wireless data transmitter 18 on the capsule 10 configured to transmit signals according to the Bluetooth data transmission protocol.

Communication between the capsule 10 and the receiver device 30 may be via a wireless data transmitter 18 on the capsule 10 configured to transmit signals according to the Bluetooth data transmission protocol.

Communication between the capsule 10 and the receiver device 30 may be via a wireless data transmitter 18 on the capsule 10 configured to transmit signals according to the Bluetooth long-range (coded-PHY) transmission protocol.

Specifically the signals transmitted according to the Bluetooth transmission protocol may be transmitted according to a post-excretion transmission mode, being a term referring to a transmission mode that does not depend upon paired status, by virtue of broadcasting data, or by virtue of initially attempting to transmit data to a couple/paired device but broadcasting data as a fallback in case coupling/pairing is unsuccessful. Broadcasting data may be executed in a handshake mode, inquiry mode, or discovery mode, in which data is broadcast by the data transmitter.

In the post-excretion transmission mode, the wireless data transmitter may initially attempt to pair to a receiver, and implement the broadcasting if the pairing attempt is unsuccessful. The pairing attempt may be an attempt to re-pair to a receiver that has previously been paired to the transmitter. Data may be transmitted according to a coded-PHY Bluetooth transmission protocol, or according to a standard Bluetooth transmission protocol.

In the post-excretion transmission mode example, excretion of the ingestible capsule device 10 from the subject may be detected by an on-board environmental temperature sensor 14, the measurements, signal, or readings of which are monitored by the on-board processor 151 which triggers the beacon transmission mode of the wireless data transmitter 18 to transmit a data transmission payload immediately upon detection of capsule excretion.

The post-excretion transmission mode may be triggered by determination that an excretion event has occurred (i.e. that the capsule has been excreted) based on readings of an on-board temperature sensor, and specifically a decrease from the in-vivo temperature. In a case in which the capsule device 10 has already been paired to a receiver 30 such as a smartphone, for example during an initiation procedure, the capsule device 10 may attempt to re-pair, and if successful, transmit a data transmission payload to the paired receiver 30. In the event of re-pair being unsuccessful, for example after a finite number of attempts or after a timeout (for example, 1 second, 3 seconds, 5 seconds), the wireless data transmitter 18 is configured to transmit a data transmission payload in a discovery, inquiry, or handshake mode, which is ordinarily a pre-cursor to pairing and enables some data transfer. A dedicated application at the receiver 30 is configured to access and process the data transmission payload so transferred.

The post-excretion transmission discussed above is exemplary of an event-triggered transmission mode. It is noted that other events may be detected by the on-board sensors and used as a trigger to begin transmission of a data transmission payload or to alter data transmission parameters.

The data transmission payload may comprise one or more from among: the diagnosis outcome (positive/negative/suspected), an indicator of severity of gastroparesis, and one or more calculated metrics or parameters leading to the diagnosis. For example, a representation of the gastroparesis indicator spike or candidate spikes, whether that representation be the underlying readings from the TCD gas sensor, or a parameter derived therefrom such as a time series of CO2 concentration, or a representation of detected spikes or candidate spikes, such as number of spikes and height of each, number of spikes and area under each, aggregate area under spikes, and/or aggregate spike height.

The term candidate spike is used to denote a feature in the readings, such as an increase in CO2 concentration values, that may or may not be considered to be a spike and is subjected to downstream processing to determine whether or not the feature is to be processed as a spike. Embodiments may be configured to distinguish spikes from other features associated with an increase in CO2 concentration values on-the-fly or in downstream processing. For example, a low-pass filter may be applied to filter out features not meeting a predefined threshold concentration increase.

Ingestible capsule devices 10 such as disclosed in Australian patent application number 2022900873 and predecessor versions thereof (all housing gas sensor apparatus inter alia other sensor devices and electronic components) are usable as ingestible capsule devices 10 in methods disclosed herein.

FIG. 4 illustrates a method.

### Obtaining a Time Series of Readings

At S401 data representing a time series of readings from gas sensor apparatus housed within a ingestible capsule device 10 orally ingested by a subject is obtained, for example at a processor 151. The time series of readings are taken during exposure of the gas sensor apparatus to a gas mixture at the ingestible capsule device 10 during passage of the ingestible capsule device 10 through a gastrointestinal tract of the subject 40. Each reading has a value representing a signal output by gas sensing apparatus that is sensitive to CO2 concentration. The readings may be taken at predefined intervals, such as every second, every 5 seconds, every 10 seconds, every 15 seconds, every 20 seconds, every 30 seconds, every minute. The readings form a time series. The readings may each include an explicit indication of time such as a time stamp, or time may be implicit by virtue of position within a chronological sequence. For example, post-initiation, the nth reading is at a time of n×m seconds, wherein m is the period between successive readings.

The gas sensor apparatus may be a single gas sensor such as thermal conductive device (TCD) gas sensor that is sensitive to CO2 concentration. The TCD gas sensor may be operated at a plurality of temperatures (i.e. driven with varying input power) to add an additional dimension to the readings, from which additional information the CO2 concentration is derivable (for example by comparison of TCD readings at different sensor temperatures). That is, CO2 concentration within the gas mixture is derivable from the variability of TCD at different TCD gas sensor operating temperature setpoints.

The processor executing the method may be on-board the ingestible capsule device 10, or off-board, wherein off-board includes being either at a receiver apparatus 30 in direct communication with the ingestible capsule device 10, or at a remote apparatus 20 in data communication with the receiver apparatus 30.

Optionally, the ingestible capsule device 10 further comprises processor hardware 151, memory hardware 152, and a wireless transmitter 18, and the processor hardware 151 in cooperation with the memory hardware 152 is configured to perform either the whole method of FIG. 4 during passage of the ingestible capsule device 10 through the gastrointestinal tract of the subject 40, or to perform steps S401 to S403 during said passage, and further to completing step S403 or S404, to transmit data indicating one or more detected spikes in the CO2 concentration or a gastroparesis diagnosis or suspected gastroparesis diagnosis to a receiver device 30 via the wireless data transmitter 18. The processor hardware 151 and memory hardware 152 may be combined in a single chip.

### DETECTING SPIKES IN CO2 CONCENTRATION AND DISTINGUISHING CAUSES

Processing steps S402 and S403 are mutually interdependent and may be performed one after the other, in any order, or concurrently. Furthermore, since the processing may be performed on-the-fly, it may be that processing steps S402 and S403 (and also S404) are performed whilst S401 is still ongoing.

At steps S402 and S403, the obtained data representing the time series of readings from the gas sensing apparatus is processed to identify spikes in CO2 concentration and to distinguish those spikes as being a gastric-duodenal transition indicator spike and gastroparesis indicator spike or spikes. The distinction may be made based on chronology, since gastroparesis indicator spikes are caused by CO2 concentration increase while the capsule 10 is resident in the stomach, whereas the gastric-duodenal indicator spike is caused by the capsule 10 passing out of the stomach and into the small intestine. Therefore, it can be appreciated that the two steps are somewhat logically interdependent, and though the gastroparesis indicator spikes may be detected first (if spike detection is performed on-the-fly rather than retrospectively), the determination that they are gastroparesis indicator spikes is dependent upon detection of a gastric-duodenal transition indicator spike in later CO2 concentration values. Alternatively the gastric-duodenal transition timing may be determined from, for example, H2 concentration readings, accelerometer readings, and/or reflectometer readings, so that based on that timing the CO2 concentration spike at gastric-duodenal transition is not mis-identified as a gastroparesis indicator spike, because it does not precede gastric-duodenal transition. Embodiments may incorporate a five-, ten-, or fifteen minute buffer into the determined gastric-duodenal transition timing, so that any CO2 concentration spike within the buffer preceding the determined gastric-duodenal transition timing is not detected as a gastroparesis indicator spike.

The processing the readings may include deriving or otherwise extracting or determining CO2 concentration values from the gas sensing apparatus readings. For example, the gas sensing apparatus readings may be from a TCD gas sensor which is operated to take readings at different operating temperature setpoints by the processor hardware 151 or some other on-board controller or microcontroller. By comparing the TCD gas sensor readings at the different operating temperature setpoints, the CO2 concentration is derivable. It is noted that other techniques for measuring CO2 concentration exist such as electrochemical sensors, non-dispersive infrared sensor and metal oxide semiconductor sensors.

At S402 the recorded sensor readings are processed to detect one or more spikes in the CO2 concentration in the gas mixture preceding a determined gastric-duodenal transition timing.

For example, a spike may be detected by identifying: a first period of increasing CO2 concentration at a rate of change with respect to time exceeding an increase gradient threshold, the increase gradient threshold being either predefined or calculated based on the subset of the time series of readings, followed by a second period of decreasing CO2 concentration at a rate of change with respect to time exceeding a decrease gradient threshold, the decrease gradient threshold being either predefined or calculated based on the subset of the time series of readings, wherein if the first period and the second period are identified, and if a duration between the identified first and second periods is below a predefined threshold, the readings representing the first period and the second period are detected as a spike. As a further example, the first derivative of the CO2 concentration with respect to time may be monitored to detect an inflection point, wherein the inflection point is determined to be indicative of a spike if it is at a height more than a threshold above a calculated (for example by a rolling average) baseline or if a gradient defined by readings preceding the inflection point is above a threshold and likewise a gradient defined by readings proceeding the inflection point is above a threshold. As a further example, a pattern matching algorithm may be configured to detect spikes in the readings, wherein the pattern matching algorithm is pre-trained with training data comprising readings containing labelled spikes and training data without spikes. The pattern matching algorithm may be a neural network such as a convolutional neural network.

The detecting one or spikes in CO2 concentration during gastric residence of the capsule 10 (i.e. preceding a spike associated with gastric-duodenal transition) may be performed by the on-board processor 151, i.e. on-the-fly, or may be determined by an off-board processor at a receiver device 30 or a processing apparatus receiving data therefrom, either in real-time or retrospectively.

### DETERMINING LOWER BOUND ON TIMING IN WHICH CO2 CONCENTRATION SPIKES ARE DETECTABLE

An ingestion event may be determined by a user recording a timing of ingestion on a user interface such as provided by an application or other software running on a receiver device 30. An ingestion event may be determined by, for example, receiving temperature readings from a temperature sensor on board the capsule 10, and determining when the temperature readings start to be within a range predefined for a subject stomach. Ingestion event may also be determined by a relative humidity sensor on board the capsule, by determining when the relative humidity readings start to be within a range predefined for a subject stomach. The determined ingestion event timing is not necessarily the start event for the gastric time period. For example, to filter out effects caused by intake and outtake of breath whilst the capsule 10 is in the oesophagus, a predefined delay (such as five minutes or more, ten minutes or more, twenty minutes or more, thirty minutes or more) is applied between determined ingestion event timing and start of the gastric time period. The ingestion event timing may be determined by the on-board processor 151, i.e. on-the-fly, or may be determined by an off-board processor at a receiver device 30 or a processing apparatus receiving data therefrom, either in real-time or retrospectively.

### DETERMINING UPPER BOUND ON TIMING IN WHICH CO2 CONCENTRATION SPIKES ARE DETECTABLE

Different techniques may be used to bound temporally the readings from the gas sensor apparatus that are processed to detect the gastroparesis indicator spikes. In a first, ICJ-based, technique, and predicated upon there being a VOC gas sensor in the gas sensor apparatus, or some other mechanism for detecting passage of the capsule 10 across the ileocecal junction, an ileocecal junction indicator is detected and used as an upper temporal bound. A second, gastric-duodenal-transition-based technique, is predicated upon there being an accelerometer 19 in the capsule, a reflectometer formed by a directional coupler in series with a transmission antenna, or some other means beyond the spike in CO2 concentration of indicating gastric-duodenal transition. In the second technique, the gastric-duodenal transition indicator is detected in one or more of the accelerometer readings, the reflectometer readings, and any other means of indicating gastric-duodenal transition, and based of the timings of the indicator or indicators being coincident with (one another and) a spike in CO2 concentration, the spike in CO2 concentration is determined to be caused by gastric-duodenal transition of the capsule 10. Thus, the said spike is taken by the processor as an upper temporal bound on the CO2 concentration values in which gastroparesis indicator spikes are detected or detectable. In either case, the lower temporal bound may be, for example, a capsule 10 ingestion event.

In other words, gastroparesis indicator spikes are spikes in CO2 concentration in the gas mixture at the capsule while the capsule 10 is resident in the stomach. But it is necessary to discount a latest spike chronologically since it is known that a spike in CO2 concentration occurs in both healthy and gastroparetic patients at the gastric-duodenal transition. Some embodiments may determine a gastric-duodenal transition timing from data other than the CO2 concentration data, so that by knowledge of that timing and its use as an upper bound the spike caused by gastric-duodenal transition is distinguishable from the gastroparesis indicator spikes. So the gastric-duodenal transition timing is determined, either based on direct determination by the gastric-duodenal-transition-based technique, or by indirect determination by the ICJ-based technique (and reasoning that the latest CO2 concentration spike preceding ICJ is caused by gastric-duodenal transition).

The purpose of the upper bound is to filter out or otherwise prevent spikes in CO2 concentration associated with presence of the capsule in the cecum and beyond being erroneously detected as gastroparesis indicators.

A gastric-duodenal transition indicator may be detected in one or more of the following sensor outputs:
If the capsule 10 includes a reflectometer formed of an antenna (which may be the antenna of the wireless data transmitter 18) in series with a directional coupler, a gastric-duodenal transition indicator may be detectable in readings of the reflectometer, for example, as a baseline shift.
If the capsule 10 includes an accelerometer such as a tri-axial accelerometer, a gastric-duodenal transition indicator may be detectable in readings of the accelerometer, for example, in a metric derived from the readings and representing agitation or angular movement of the capsule 10, based on an observation that capsule 10 is more agitated or exhibits a higher rate of angular movement post-gastric emptying.
A gastric-duodenal indicator may be detectable in readings of the gas sensing apparatus, for example in the output signal of a TCD gas sensor, either in the raw output signal or in calibrated readings representing concentration of one or more constituent gases such as CO2, such an indicator is distinguishable from gastroparesis indicators by occurring chronologically later than the gastroparesis indicators.

Processing to detect or identify a gastric-duodenal transition indicator includes monitoring or otherwise assessing recorded readings from the TCD gas sensor, the accelerometer, and/or the reflectometer, to detect a characteristic feature in the readings that may indicate gastric-duodenal transition of the capsule 10. Characteristic features may be spikes, baseline shifts, inflection points, depending on the sensor and the nature of the readings.

Since the timing of gastric emptying (gastric-duodenal transition) may be determined by sensors such as the accelerometer 19 and the reflectometer 18, it is not strictly necessary to detect the spike in CO2 concentration at S403, as long as the timing of the spike can be determined. That is, if the timing of gastric-duodenal transition of the capsule 10 is determined, then step S402 may only detect spikes in readings preceding that timing, so that the CO2 concentration spike associated with the gastric emptying event is not determined.

Determining whether an indicator is caused by a gastric-duodenal transition event of the capsule 10 may comprise calculating a confidence score for the hypothesis that the detected indicator was caused by the said gastric-duodenal transition event. Optionally, a threshold may be applied to the confidence score wherein exceeding the threshold is a positive determination. A confidence score below the threshold may be a negative determination or may be a trigger for further processing such as processing the readings of sensors other than that providing the detected indicator to identify one or more further indicators (for example, if the initial sensor is in the TCD gas sensor output then processing the accelerometer and/or reflectometer readings). A revised confidence score is then calculated based on the combination of the initial indicator and the one or more further indicators, which is compared with the threshold and a positive determination made in the event that the threshold is met.

The gastric-duodenal transition event timing may be determined by the on-board processor 151, i.e. on-the-fly, or may be determined by an off-board processor at a receiver device 30 or a processing apparatus receiving data therefrom, either in real-time or retrospectively.

### DETECT OR DIAGNOSE GASTROPARESIS BASED ON SPIKES

At S404 a processor housed either on-board the capsule 10 or at a receiver device or processing apparatus in data communication therewith is configured to determine whether or not the spikes detected at S403 indicate that the subject is suffering from gastroparesis. In other words, at S404 a diagnosis of gastroparesis, suspected gastroparesis, or a negative diagnosis, is made, based on the detected gastroparesis indicator spike or spikes. For example, S404 may comprise calculating a confidence score in the diagnosis or suspected diagnosis, the confidence score being calculated with reference to the detected one or more spikes relative to one or more reference cases. A lookup table may be stored on a memory (such as on-board memory hardware 152) accessible to the processor executing S404 which lookup table may store a confidence score value for a number of detected spikes as a key. In a further example, the lookup table may be multi-dimensional and may store a confidence score value for a key of up to n components, wherein each component is a spike height or some other value representing a magnitude of each detected gastroparesis indicator spike in CO2 concentration. Alternatively, a formula may be stored enabling a confidence score to be calculated for input values including number of detected spikes and optionally also height per spike, or some other metric indicating magnitude of each detected gastroparesis indicator spike in CO2 concentration. As a further alternative, it may be that, in place of confidence score, a simple single Boolean value indicating a positive or negative value is calculated and output (for example in a report transmitted away from the capsule 10 by a wireless data transmitter 18). As a further alternative, a single value may indicate one of three outcomes: gastroparesis diagnosis, suspected gastroparesis diagnosis, negative gastroparesis diagnosis. Wherein suspected gastroparesis diagnosis may be a signal or alert to a clinician to undertake further testing or investigation. A confidence score is a quantification of the likelihood that the hypothesis “the detected gastroparesis indicator spikes are caused by gastroparesis in the subject” is true.

The lookup table is exemplary of a model. The model may also be one or more functions, a machine learning model, or some other processing model. In either case, the model is configured, based on calculated values of one or more input factors or input parameters, to generate a corresponding output being a quantitative or qualitative indication of likelihood of gastroparesis being present in the subject. Furthermore, the output may include an indication of severity of gastroparesis, or likely severity of gastroparesis.

Examples of input factors include:
a count of the number of gastroparesis indicator spikes;
a count of the number of gastroparesis indicator spikes and the height of each gastroparesis indicator spike;
a histogram or another representation of distribution of heights of each gastroparesis indicator spike;
an aggregate height of the gastroparesis indicator spikes;
a count of the number of gastroparesis indicator spikes and the area under each gastroparesis indicator spike;
a histogram or another representation of distribution of areas under each gastroparesis indicator spike;
an aggregate area under the gastroparesis indicator spikes;
temporal duration between ingestion of the ingestible capsule device and the gastric-duodenal transition timing of the ingestible capsule device.

The model is predefined in a model configuration process, which may be, for example, a training stage of a machine learning model, or manual configuration of a model by an expert, or some other configuration of the model based on sample or training data. The training data being, for real life subjects ingesting a capsule to generate data from which values of one or more of the input parameters are calculated, the values of the one or more input parameters, along with a target output being a positive or negative diagnosis and/or an indication of severity. By training or otherwise configuring the model based on a number of subjects, being, for example, 20 or more, 30 or more, 40 or more, 50 or more, 100 or more, the model is taught or otherwise configured to predict a positive or negative diagnosis and/or an indication of severity based on values of one or more input factors. The model may be a machine learning model such as an artificial neural network. In a further example, the model may be a threshold value applied to a single calculated input factor (for example, if aggregate area under the spikes exceeds a defined threshold, then positive diagnosis), or a plurality of thresholds for respective individual input factors which must all be satisfied or a predefined proportion must be satisfied for a positive diagnosis. Such a threshold or thresholds may be determined by a human expert analysing sample data and defining a threshold based on the sample data. In the case of the machine learning model or the one or more defined thresholds, an additional criterion may be a minimum temporal duration between ingestion timing and gastric-duodenal transition timing. For example, embodiments may be configured with a minimum-time-to-gastric-emptying threshold of 4 or 5 hours (or anywhere between), wherein a requirement for a positive diagnosis is that the minimum-time-to-gastric-emptying threshold is met. Optionally, said minimum-time-to-gastric-emptying threshold not being met, but one or more other thresholds being met, may lead to a diagnostic result of suspected gastroparesis output, rather than a positive diagnosis per se.

Alternatively, the temporal duration between ingestion and determined gastric-duodenal transition of the capsule is determined, and if it is within a predefined window (such as between 4 and 5 hours), then the above one or more thresholds applies to an input factor relating the gastroparesis indicator spikes is applied, and if satisfied, then the model output is suspected gastroparesis, or a positive diagnosis, and if not satisfied, then the model output is negative diagnosis. That is, the model may be an algorithm comprising one or more conditions, the one or more conditions being based on a threshold applied to a value of an input factor relating to the gastroparesis indicator spikes, and/or a minimum-time-to-gastric-emptying threshold. Examples of input factors include: a count of the number of gastroparesis indicator spikes; a count of the number of gastroparesis indicator spikes and the height of each gastroparesis indicator spike; a histogram or another representation of distribution of heights of each gastroparesis indicator spike; an aggregate height of the gastroparesis indicator spikes; a count of the number of gastroparesis indicator spikes and the area under each gastroparesis indicator spike; a histogram or another representation of distribution of areas under each gastroparesis indicator spike; an aggregate area under the gastroparesis indicator spikes; temporal duration between ingestion of the ingestible capsule device and the gastric-duodenal transition timing of the ingestible capsule device.

At S404, a determination is made as to whether the CO2 concentration data contains evidence of gastroparesis, and whether that evidence is sufficient to positively diagnose gastroparesis in the subject. In addition, a qualitative or quantitative indication of severity of gastroparesis in the patient may be determined.

### HEALTHY PATIENT SIGNALS EXAMPLE DATA

FIG. 5 illustrates sensor signals and measurements derived from sensor signals generated by a ingestible capsule device 10 during passage through the gastrointestinal tract of a subject. FIG. 5 illustrates CO2 concentration values, which is a value that may be calculated based on TCD gas sensor readings as calibrated by temperature readings. Other techniques for measuring CO2 concentration include direct measurement via a dedicated CO2 sensor. Furthermore, it is noted that further data from which to calculate CO2 concentration may be obtained by taking readings from a heater side of a VOC gas sensor, thereby producing TCD data. FIG. 5 illustrates temperature readings, which are direct measurements by the environmental temperature sensor 14a. FIG. 5 illustrates hydrogen concentration values, which is a value calculated based on the TCD gas sensor readings. FIG. 5 illustrates relative humidity values, which are direct measurements by the relative humidity sensor 14b. FIG. 5 illustrates normalized pitch values, which is a metric calculated based on the accelerometer sensor 19 signal and which may be referred to as angle travelled (discussed in more detail below). FIG. 5 illustrates direct coupler output values, which are direct measurements of the reflectometer formed by the antenna and the directional coupler. In each case, the values are illustrated as a time series. Embodiments do not require all of the values illustrated in FIG. 5, as long as the CO2 concentration is derivable.

It is noted that both CO2 concentration and H2 concentration are derivable from TCD gas sensor readings. The gases are distinguishable from one another by techniques including measuring TCD at different operating temperatures, and deriving concentrations of constituent gases based on the variation between TCD gas sensor measurements at the different operating temperatures. The processor 151 or some other controller or microcontroller may control the variation of the operating temperature setpoint of the TCD gas sensor. It is further noted that ingestible capsule devices including VOC gas sensors may be configured to measure thermal conductivity by measuring resistance from a heater side of the VOC gas sensor. Further information on distinguishing between constituent gases and driving individual gas sensors is provided in PCT/AU2017/000167.

FIG. 5 illustrates a time series of CO2 concentration level values in which there is no spike during a gastric time period during which the capsule 10 is resident in the stomach of the subject. A single spike in CO2 concentration is detectable, but since this spike is associated with gastric duodenal transition event it is not considered to be during the gastric time period. In other words, it indicates the end of the gastric time period. The single spike is associated with the gastric duodenal transition event. As discussed above, the association of the single spike with the gastric-duodenal transition event may be based on direct determination by the gastric-duodenal-transition-based technique (in which readings in signals from reflectometer 18 and/or accelerometer 19 provide gastric-duodenal transition indicators), or by indirect determination by the ICJ-based technique (and reasoning that the latest, or in this case only, CO2 concentration spike preceding ICJ is caused by gastric-duodenal transition). It is noted that capsules may be configured to execute both techniques and to cross-reference one another, wherein if the two techniques do not agree a flag or error may be included in a report output by the capsule via the wireless data transmitter for review by a clinician.

### GASTROPARESIS PATIENT SIGNALS EXAMPLE DATA

FIGS. 6 & 7 are comparable with FIG. 5 in terms of the illustrated time series. Food and drink events are marked, which may be detected by a user interaction with a user interface such as on a receiver apparatus 30 receiving signals from the capsule 10, or may be detected by monitoring the environmental temperature signal while the capsule 10 is in the stomach, with changes in temperature in the stomach being determined to have been caused by ingestion of food or drink.

In the example of FIGS. 6 & 7, steps S402 and S403 may be performed by the ICJ-based technique, that is, detecting an ICJ transition indicator in the data, from which a determination of gastric-duodenal transition timing is made by identifying the last spike chronologically as a gastric-duodenal indicator spike, and any preceding spikes in the CO2 concentration data as gastroparesis indicator spikes (in this example a very evident rate of change of VOC gas sensor readings is detectable). FIGS. 6 & 7 are data from a live test with a different patient or subject in each case.

In the case of FIG. 6, two spikes are detected in the CO2 concentration values preceding the spike associated with gastric-duodenal transition. It is noted that there are some perturbations preceding the first spike that may fulfil one or more of the criteria for detection as a spike, but which may have been filtered out, for example because the magnitude of increase in CO2 concentration that they represent does not meet a threshold.

In the case of FIG. 7, five spikes are detected in the CO2 concentration values preceding the spike associated with gastric-duodenal transition. It is noted that some of the detected spikes may, in some configurations of the processing, have been filtered out, for example because the magnitude of increase in CO2 concentration that they represent does not meet a threshold.

In the processing at S404, a positive or negative diagnosis of gastroparesis is made, or in some embodiments a diagnosis of suspected gastroparesis may be included in the possible outcomes. Optionally, the diagnosis may be of suspected gastroparesis, in other words, the processing output may be an indication that gastroparesis is present in the subject, but that owing to the clinical complexities associated with gastroparesis and/or best practice regarding gastroparesis diagnosis, the indication is considered to represent suspected gastroparesis rather than gastroparesis. Embodiments may base diagnosis (of gastroparesis, or suspected gastroparesis) on a confidence score representing a likelihood of the hypothesis that the detected spikes at S403 are caused by gastroparesis. The confidence score may be based on a probability distribution represented in a lookup table, as a function, or otherwise, providing an output confidence score based on a one dimensional input being a number of detected spikes at S403 or a multi-dimensional input including a metric representing each spike, for example, spike height in terms of absolute or proportional increase in CO2 concentration per spike. Embodiments may also output an indicator of severity of gastroparesis. Such an indicator may comprise or may be based upon a metric such as aggregate area under gastroparesis indicator spikes.

### ACCELEROMETER DATA

The accelerometer data illustrated in FIGS. 5 to 7 is not a direct measurement from the accelerometer sensor 19. Rather, the accelerometer data illustrated in FIGS. 5 to 7 is a metric calculated from the accelerometer data and representing agitation of the capsule 10. The metric may be calculated on-board the capsule 10 by the processor hardware 151 executing instructions stored on the memory hardware 152, or the raw signal may be stored and transmitted by the wireless data transmitter 18 for processing by a receiver apparatus 30 or a remote processing apparatus in data communication therewith. Sample metrics are detailed below by way of example:

The capsule 10 orientation may be measured using a triaxial accelerometer 19 and tracking the gravity vector with respect the capsule frame of reference. When the capsule 10 leaves the stomach it tends to experience rapid changes in its orientation as it transits through the duodenum and small intestine. “Angle Travelled”, simply accumulates the orientation change in excess of a 90 degree hysteresis angle. This technique tends to be robust to small changes in orientation experienced in the stomach and avoids some of the complexities of other approaches.

An example technique for processing accelerometer data may be referred to as angle travelled. Angle travelled uses vector mathematics to calculate the angle between the gravity vector and a temporary vector. The temporary vector is pulled in the direction of the change in angle, only when this angle exceeds a given threshold (currently 90 Deg). It is then the accumulation of the change in the temporary vector that is visualized in the representation from which markers are identifiable. What is generally seen is that this measure does not change much in the stomach since the angle between the gravity and temporary vectors rarely exceed the threshold in any one direction, (small back and forth orientation changes in the stomach are effectively ignored by the inherent hysteresis of this algorithm) and that once in the tortuous lumen of the small intestine, this measure accumulates significantly due to the larger, more continuous orientation changes of the capsule. Thus, a step change in the cumulative angle travelled measure is a gastric-duodenal transition indicator.

In an exemplary implementation of angle travelled: the accelerometer readings may provide a reading of an orientation of the ingestible capsule relative to a frame of reference in fixed relation to a gravitational vector. Processing of the readings from the accelerometer may comprise recording an orientation of the ingestible capsule given by a first accelerometer reading as a reference orientation, and repetitively in respect of each successive accelerometer reading chronologically: determining whether the orientation of the ingestible capsule given by the respective accelerometer reading is more than a threshold angular displacement from the reference orientation, and if the threshold angular displacement is not met, progressing to the next accelerometer reading without changing the reference orientation, and if the threshold angular displacement is met, changing the reference orientation to align with the orientation of the ingestible capsule given by the respective accelerometer reading. An indicator, such as the gastric-duodenal transition indicator, may be a step change in the rate of change of the reference orientation.

As illustrated in FIG. 5, a step change in a plot of angle travelled is identifiable within a threshold time period of a detected spike in the TCD gas sensor readings. Therefore, the step change in the plot of angle travelled increases confidence in the hypothesis that the detected spike in the TCD gas sensor readings is caused by gastric-duodenal transition. There are two approximately contemporaneous gastric-duodenal transition indicators, which enables the timing of one of the indicators (which one may be pre-selected, for example, the TCD gas sensor readings) to be determined as the timing of the transition event.

A second exemplary technique for processing accelerometer data may be referred to as total roll. Total roll calculates the angle between the gravity vector and each of the capsule X, Y and Z axes and expresses this as a continuous measure that can accumulate beyond 360 Deg. For example, if the capsule x axis is at an angle of 350 Deg and rotates by a further 20 Deg, the resulting angle is expressed as 370 Deg rather than 10 Deg. This helps when representing the readings as a plot from which markers are identified since it avoids the sudden angle changes associated with crossing the zero line. In the example a real change of 20 Deg would be visualized instead of an artificial change of 340 Deg. In addition to this basic approach, low pass filtering may be applied to filter the raw data to remove sensor noise. Additionally, angles are only calculated when the raw accelerometer data provide sufficient data to calculate a meaningful angle. An example of where this is not the case is when the two accelerometer axis values used to calculate the orientation angle around the third axis both approach zero. In this case the calculation will be dominated by sensor noise and so a meaningful angle cannot be determined.

The raw accelerometer signal provides a reading of an orientation of the ingestible capsule relative to a frame of reference in fixed relation to a gravitational vector. Exemplary processing of the readings from the accelerometer may comprise for each of three orthogonal axes in fixed spatial relation to the ingestible capsule derivable from the reading of the orientation, repetitively in respect of each successive accelerometer reading chronologically: calculating, as a scalar value, a change in the orthogonal axis relative to the gravitational vector from the preceding accelerometer reading; applying a low pass filter to the calculated changes; recording the cumulative filtered calculated changes. A marker serving as a gastric-duodenal transition indicator may be, for example, an increase (such as a spike or step change) in the rate of increase in the cumulative filtered calculated changes, said increase being above a predefined threshold.

The following disclosure describes techniques for determining gastric duodenal transition timing, which may be included in methods, in particular methods employing the gastric-duodenal-transition-based technique for setting an upper bound on the timing of readings in which gastroparesis indicator spikes are detectable. It is noted that embodiments do not necessarily detect a gastric-duodenal transition indicator in the CO2 concentration data (although they may do so). Rather, embodiments are configured to determine the timing of the gastric-duodenal transition (of the capsule 10) so that an upper-or later-bound may be applied to the CO2 concentration data in which spikes are detected or classified as gastroparesis indicator spikes.

Since embodiments are distinguishing between CO2 concentration spikes caused by gastroparesis and those caused by gastric-duodenal transition, it may be that gastric-duodenal transition is determined by cross-checking with other sensor outputs.

However, it is noted that by detecting ileocecal junction transition, for example by identifying an increase in concentration of volatile organic compounds sensed by an on-board VOC gas sensor (indicated as ICJ in FIG. 5), a bound can be set on a time period in which spikes are detectable in the CO2 concentration values, and if a single spike is detected between ingestion event timing and ileocecal junction transition timing, then that spike is identifiable as a gastric-duodenal transition indicator, whereas if plural spikes are detected, then the chronologically latest is identifiable as a gastric-duodenal transition indicator. Once the gastric-duodenal transition indicator spike is identified, a later-bound is set on the subset of CO2 concentration values in which spikes are detectable for use in gastroparesis diagnosis. In other words, the latest spike chronologically is discounted, and any earlier spikes are detected as spikes in S403. In FIG. 5, on the other hand, it can be appreciated that once the ileocecal junction transition is identified (see ICJ in FIG. 5), there is only a single CO2 concentration spike between ingestion and ileocecal junction transition, which enables a processor to determine that the CO2 concentration spike is caused by gastric-duodenal transition and therefore is not during the gastric residence time period of the capsule 10 and is not detectable as a spike in S403.

FIG. 6 illustrates the same selection of time series values as illustrated in FIG. 5, but from a different subject. The ileocecal junction indicator is marked as ICJ in FIG. 6, the sharp increase in VOC gas sensor readings. Embodiments may maintain a count of detected spikes for inclusion in a report transmitted away from the capsule 10 to a receiver device by one or data transmission techniques described above.

### Detecting Gastric-Duodenal Transition and Ileocecal Junction Transition

Recorded readings later than the determined ingestion event timing are analysed to determine timing of gastric-duodenal transition.

The gastric-duodenal transition event is the capsule 10 gastric emptying or crossing the interface between the stomach and the duodenum. Candidate gastric duodenal indicator or indicators may be detected in a gastric-duodenal indicator subset of recorded readings (noting that this subset is conceptually distinct from the subset defining the gastric time period, but that the two may overlap partially or completely both temporally and in terms of sensors), the gastric-duodenal indicator subset being defined temporally by starting after an ingestion event. Furthermore, the gastric-duodenal indicator subset may be constrained by sensor, comprising readings from the TCD gas sensor 131. The gastric-duodenal indicator subset may further comprise readings from the reflectometer (i.e. the antenna 17 and directional coupler 171) and/or the accelerometer 19.

The candidate gastric-duodenal transition indicator in the TCD gas sensor readings may be a, spike, step change or an inflection point in the TCD gas sensor readings. A correction may be applied to the TCD gas sensor readings to account for changes in environmental temperature, based on recorded readings from the environmental temperature sensor 14a. The correction may be applied at the detecting stage so that the recorded readings themselves are corrected to account for changes in environmental temperature, and a candidate gastric-duodenal transition indicator is detected in the corrected readings. Alternatively, the candidate gastric-duodenal transition indicator may be detected in the raw readings (i.e. the uncorrected readings) and then at the determining step a check performed to determine whether or not the indicator is attributable to a change in the environmental temperature or not, and if not, then a further condition is applied in the determination (for example, recorded readings from another sensor are checked for a contemporaneous indicator), or the ICJ transition timing relative to the candidate gastric-duodenal transition indicator and any other spikes in CO2 concentration is used in the determination.

The primary physical mechanism being sensed in the TCD gas sensor readings in detecting the gastric-duodenal transition indicator is as follows: Hydrochloric acid in the gastric juices leaving the stomach mixes with bicarbonate within the bile acids that is released by the pancreas. This bile acid works to neutralize the pH of the liquid and a by-product of this reaction is CO2. In this area of the GI tract the surrounding gases are primarily N2 and 02 with some trace amounts of CO2. The amount of CO2 created in this reaction are significantly higher than the trace amounts that are around due to swallowing of exhaled breath. Therefore, simply using the TCD sensor output without calculating CO2 is appropriate. In other words, the TCD gas sensor readings, once corrected for environmental temperature variations, themselves provide the gastric-duodenal transition indicator, owing to a change in heat conductivity caused by variation in CO2 concentration across the two sides of the gastric-duodenal transition. For motility purposes (i.e. for determining the gastric-duodenal transition timing) there is no particular need to calculate the actual CO2 concentration.

As the TCD sensor 131 is affected by the temperature of the gas mixture at the location of the capsule, a temperature correction process is required to account for changes in the external environmental temperature changes i.e. drinking cold water, exercise, eating etc. Starting from the determined ingestion event timing, the first bump, step change or large inflection in the readings of the TCD gas sensor 131 plotted against time, that is not associated with an environmental temperature change, identifies the gastric-duodenal transition, or gastroparesis. Distinguishing between the two is discussed in more detail elsewhere in the present disclosure.

FIG. 8a illustrates recorded readings of an environmental temperature sensor 14a (top line of readings on the top graph) against time, and corrected TCD gas sensor readings against time for an instance of capsule ingestion and progression through a GI tract. The candidate gastric-duodenal transition indicator, which may be labelled gastric emptying, is indicated by a spike above a threshold height in the corrected TCD gas sensor readings. Spike height may be measured, for example, by distance (e.g. as a proportion, as an absolute value, or as a number of standard deviations) from a trend line fitted against the readings up to that point, or from an average value up to that point (wherein the processor maintains an average value).

In the present implementation, since a detected spike may be a gastric-duodenal transition indicator or a gastroparesis indicator (in other words the cause of the spike may not necessarily be determined to by gastric-duodenal transition of the capsule 10), further information is required to determine gastric-duodenal transition timing. The further information may be provided by cross referencing with signals from other sensors including reflectometer and/or accelerometer, or by ICJ detection and an assumption that the final CO2 concentration spike preceding ICJ is the gastric-duodenal transition indicator.

FIG. 8B shows gastric emptying as visible in TCD sensor output and CO2 readings. CO2 is produced when the hydrochloric acid in the gastric juices leave the stomach and mix with bicarbonate in the bile acids released by the pancreas. This reaction also neutralizes the pH of the liquid. Embodiments use the temperature compensated raw TCD sensor output to detect this event, rather than the calculated CO2, since it contains much less noise. The TCD sensor output is adjusted to compensate for the temperature fluctuations measured by the environmental temperature sensor 14a. An algorithm is used to find the moment CO2 increases by removing drinking events and searching for one or more distinct discontinuities in the TCD output between ingestion and ICJ transition.

The on-board processing may include detecting, as a candidate gastric-duodenal transition indicator, a gastric-duodenal transition indicator in the TCD gas sensor readings from the gastric-duodenal indicator subset of recorded readings. And the on-board processing may further include making a determination as to whether or not the detected gastric-duodenal indicator is caused by gastric-duodenal transition or not, and in particular therefore providing a basis for distinction from the gastroparesis indicators.

The determination processing may include detecting whether or not a second gastric-duodenal transition indicator is present in readings from the first subset other than the TCD gas sensor readings and contemporaneous with the first gastric-duodenal transition indicator, and if the second gastric-duodenal transition indicator is detected, determining that the first transition event has occurred and a timing thereof based on a timing of the first gastric-duodenal transition indicator.

Readings contemporaneous with the candidate gastric duodenal transition indicator from other sensors or pseudo sensors are analysed to identify one or more second gastric-duodenal transition indicators. The temporal bounds of the readings included in the analysis may be, for example, a predefined temporal distance either side of the first gastric duodenal transition indicator, for example, one second, five seconds, ten seconds, twenty seconds, thirty seconds, one minute, two minute, or five minutes. Recorded readings from either or both of the reflectometer (i.e. the antenna 17 and directional coupler 171 configured as a reflectometer sensing whether and how the dielectric of the environment surrounding the capsule 10 changes) and the accelerometer 19 (i.e. sensing whether and how the capsule rate of orientation change varies) may be processed in seeking to identify the one or more second gastric-duodenal transition indicators.

As illustrated in FIG. 2, the circuitry includes a directional coupler 171 in series with the antenna 17, which operate as a reflectometer. A diode detector measures the amplitude of reflected signals from the antenna. The measurements of the diode detector are the reflectometer readings, and measure the reflected energy from the antenna, i.e. energy that was not radiated from the antenna 17 due to impedance mismatches. The reflectometer readings measure the antenna's radiation efficiency which is affected by the dielectric of the material surrounding the capsule

The readings may become noisy and/or a baseline shift occurs at the timing of the gastric-duodenal transition event. For example, the increase in noise and/or the baseline shift are detectable as transition indicators.

FIG. 9D illustrates (on the uppermost plot on the lower of the two sets of axes) reflectometer readings against time (labelled “Ant” for antenna), and is marked with the gastric emptying event. The antenna 17 and directional coupler 171 function as a reflectometer to measure the reflected energy from the antenna, i.e. energy that wasn't radiated out of the antenna. This signal varies as the surrounding dielectric properties change, most notably when the capsule leaves the cavernous fluid filled stomach and transitions to being surrounded by tubular tissue in the small intestine. A shift in the reflectometer readings is observed to be coincident with the TCD marker, which may be taken as confirmation that a candidate gastric-duodenal transition indicator is caused by gastric-duodenal transition of the capsule 10.

FIG. 9A is a plot of recorded readings (or processed versions thereof) against time for a number of sensors and pseudo sensors in the capsule 10. A gastric emptying (gastric-duodenal transition) event is labelled. The top plot in the graph of FIG. 9A is reflectometer readings against time (labelled “Ant” for antenna). It can be seen that a baseline shift occurs at a time coincident with the spike in corrected TCD gas sensor readings. The readings of the reflectometer may be analysed to detect a baseline shift coincident with the spike. For example, a baseline shift may be detected by, on a progressive/rolling basis, comparing a mean value of a latest number (e.g. five, ten, or twenty) of consecutive readings, with a mean value of a number of readings preceding (or proceeding in the case of reverse chronological processing) the latest number of consecutive readings. A baseline shift may be indicated by a difference more than a threshold, wherein the threshold may be an absolute value, a proportion, or determined relative to a standard deviation in the readings. Detecting a coincidental gastric-duodenal indicator in the output of the reflectometer may be sufficient to confirm that the candidate gastric duodenal transition indicator is caused by gastric-duodenal transition of the capsule 10 and thus to determine the timing of the gastric-duodenal transition. With this determination, any spikes bursts or other discontinuities in CO2 concentration values between ingestion and determined gastric-duodenal transition timing may be detected as gastroparesis indicators.

Alternatively, the combination of the two indicators may be assessed via a probability model to revise the confidence score and compare the revised confidence score with a threshold, wherein meeting the threshold is to determine that the first gastric duodenal transition indicator is caused by gastric-duodenal transition of the capsule 10 and thus to determine the timing of the gastric-duodenal transition.

### Ileocecal Junction Transition Timing

Some embodiments include detection or determination of ileocecal junction transition as part of determination of gastric-duodenal transition timing. Ileocecal junction transition timing is timing of passage of the capsule 10 through the ileocecal junction. Ileocecal junction transition indicator (or ileocecal junction indicator) or indicators may be detected in readings from the sensor side of the VOC gas sensor 132a.

The ileocecal junction transition indicator in the VOC gas sensor readings may be a spike, step change or an inflection point in the VOC gas sensor readings. Spike may be detectable via comparison of a most recent signal reading with an average-to-date value, wherein a predefined number of adjacent readings exceed one another and exceed the average-to-date by more than a predefined threshold is defined as a spike, for example. An inflection point is detectable by monitoring gradients and identifying when a second derivative (i.e. rate of change of gradient) changes from positive to negative or vice-versa. A step change may be detectable via comparison of a most recent signal reading with an average-to-date value, wherein a predefined number of adjacent readings exceed the average-to-date by more than a predefined threshold is defined as a spike, for example.

The gas environment change between the small and large intestine is significant due to the large intestine's bacterial population occurring in significantly higher prevalence, driving the creation, or increase, in volatiles and a reduction on 02 through fermentation of carbohydrates and proteins by the microbiota.

The VOC gas sensor output 132 from the sensor side 132a is sensitive to many different volatile analytes with the largest response being due to H2, and O2. At the time of transition through the ileocecal valve a large reduction on the VOC sensor is observed. As the capsule transits the GI tract the environment is increasingly anaerobic as the O2 is consumed by bacteria. FIG. 8C illustrates indicators of ICJ on plots of VOC sensor output and determined H2 concentration. The indicator in the VOC sensor output may be identified at S106a through plotting the differential of the VOC sensor side readings vs time whilst the sensor is heated and finding the tallest negative peak. This differential locates the point of greatest change which is associated with the transition but does not occur at the start of the transition event. The start of the transition event may be found by the initial inflection point from the baseline in the first derivative. Thus, the indicator may detected by the tallest negative peak, and the event timing determined by the inflection point. The tallest negative peak may be found retrospectively by analyzing VOC gas sensor readings preceding the determined excretion event timing (in the case of reverse-chronological processing). Alternatively, a threshold negative peak size may be determined, with the first peak exceeding the threshold size being detected as the ileocecal junction transition indicator.

As illustrated in FIG. 8D, an ICJ indicator is also present in the determined H2 concentration percentage, as a sharp increase in H2 when the capsule reaches the colon. The H2 produced in the GI tract is a byproduct of fermentation. The colonies of bacteria are orders of magnitude larger in the colon than in the small bowel. Therefore, determined H2 concentration may be used to add confidence to the ileocecal junction transition indicator in the VOC sensor output. H2 concentration may be sensed directly, such as by a dedicated H2 gas sensor, or may be derived from gas sensors, for example by taking TCD gas sensor readings at different operating temperature setpoints.

Sensor capsules such as that disclosed in EP3497437A1 house gas sensors and other sensors within an ingestible capsule so that readings may be made from within the gastrointestinal (GI) tract of a mammal, from which readings information about the GI tract may be determined, such as motility reports and concentrations of analyte gases.

A process for determining type and concentration of particular gases in a multi-gas mixture based on readings taken from within the GI tract by gas sensors on-board an ingestible capsule is disclosed in EP3619526A1.

1.-49. (canceled)

50. An ingestible capsule device comprising:
an ingestible indigestible bio-compatible housing;
and, within the housing:
a power source;
sensor hardware including gas sensing apparatus;
processor hardware;
memory hardware; and
a wireless data transmitter;
the memory hardware storing processing instructions which, when executed by the processor hardware, cause the processor hardware to perform a process comprising:
obtaining data representing a time series of readings from the gas sensing apparatus, the time series of readings being taken during exposure of the gas sensing apparatus to a gas mixture at the ingestible capsule device during passage of the ingestible capsule device through a gastrointestinal tract of a subject, the subject having orally ingested the ingestible capsule device, each reading having a value, the values of the readings being sensitive to CO2 concentration in the gas mixture;
processing the readings to detect one or more medical condition indicator spikes in the CO2 concentration with respect to time, a medical condition indicator spike being a spike in the CO2 concentration with respect to time at a timing after an ingestion timing of the ingestible capsule device and preceding a gastric-duodenal transition timing of the ingestible capsule device.

51. The ingestible capsule device of claim 50, wherein the process further comprises:
based on the detected one or more medical condition indicator spikes in the CO2 concentration with respect to time, diagnosing a medical condition.

52. The ingestible capsule device according to claim 51, wherein the medical condition is gastroparesis or suspected gastroparesis.

53. The ingestible capsule device according to claim 50, wherein
the gas sensing apparatus includes a thermal conductivity detector, TCD, gas sensor, wherein processing the readings from the subset to detect one or more spikes in the CO2 concentration values with respect to time includes referencing calibration data transforming TCD gas sensor reading values to CO2 concentration values.

54. The ingestible capsule device according to claim 50, wherein the process further comprises:
controlling the TCD gas sensor to multiple operating temperature set points at which to make readings, wherein processing the readings from the subset to detect one or more medical condition indicator spikes in the CO2 concentration includes comparing the TCD gas sensor readings at different operating temperature set points with one another to calculate CO2 concentration values.

55. The ingestible capsule device according to claim 52, wherein
diagnosing gastroparesis or suspected gastroparesis based on the detected one or more gastroparesis indicator spikes in the CO2 concentration with respect to time comprises:
predicting presence or absence of gastroparesis in the subject by calculating values of each of one or more factors and inputting the calculated values to a predefined model outputting a quantitative or qualitative indication of likelihood of gastroparesis being present in the subject based on the one or more input calculated values;
the calculated values of each of one or more factors comprising one or more from among:
a count of the number of gastroparesis indicator spikes;
a count of the number of gastroparesis indicator spikes and the height of each gastroparesis indicator spike;
a histogram or another representation of distribution of heights of each gastroparesis indicator spike;
an aggregate height of the gastroparesis indicator spikes;
a count of the number of gastroparesis indicator spikes and the area under each gastroparesis indicator spike;
a histogram or another representation of distribution of areas under each gastroparesis indicator spike;
an aggregate area under the gastroparesis indicator spikes;
temporal duration between ingestion of the ingestible capsule device and the gastric-duodenal transition timing of the ingestible capsule device; and
wherein the output of the predefined model includes an indication of severity of gastroparesis in the subject.

56. The ingestible capsule device according to claim 50, wherein
the ingestible capsule device also houses an environmental temperature sensor to detect an environmental temperature at the ingestible capsule device, wherein the process further comprises preprocessing the readings from the gas sensing apparatus to compensate for changes in the environmental temperature.

57. The ingestible capsule device according to claim 50, wherein
processing the readings to detect one or more medical condition indicator spikes, comprises:
detecting a gastric-duodenal transition indicator in the time series of readings from the gas sensing apparatus, determining that the gastric-duodenal transition indicator in the time series of readings from the gas sensing apparatus is caused by a gastric-duodenal transition by the ingestible capsule device, and determining a timing of the gastric-duodenal transition indicator as the gastric-duodenal transition timing.

58. The ingestible capsule device according to claim 50, wherein
processing the readings to detect one or more medical condition indicator spikes comprises:
determining an upper bound on the gastric-duodenal transition timing by positively detecting residence of the ingestible capsule device in the intestines of the gastrointestinal tract;
detecting a chronologically latest spike in the CO2 concentration with respect to time preceding the upper bound as a gastric-duodenal transition indicator spike and determining that the gastric-duodenal transition indicator spike is caused by a gastric-duodenal transition by the ingestible capsule device, and determining a timing of the gastric-duodenal transition indicator as the gastric-duodenal transition timing, and
wherein positively detecting residence of the ingestible capsule device in the intestines of the gastrointestinal tract includes detecting an ileocecal junction transition indicator, determining that the ileocecal junction indicator is caused by an ileocecal junction transition of the ingestible capsule device, and determining a timing of the ileocecal junction transition indicator as the upper bound on the gastric-duodenal indicator timing.

59. The ingestible capsule device according to claim 50, wherein
the gas sensing apparatus includes a VOC gas sensor and the ileocecal junction transition indicator is a feature in a time series of readings from the VOC gas sensor, the reading being a turning point, a step change, or a period of gradient increase exceeding a gradient increase threshold.

60. The ingestible capsule device according to claim 50, wherein the process further comprises:
obtaining data representing a time series of readings from a reflectometer housed within the ingestible capsule device and formed of an antenna in series with a directional coupler;
determining the gastric-duodenal transition timing by:
detecting a gastric-duodenal transition indicator in the time series of readings from the reflectometer, determining that the gastric-duodenal transition indicator in the time series of readings from the reflectometer is caused by a gastric-duodenal transition by the ingestible capsule device, and determining the gastric-duodenal transition timing based on the timing of the detected gastric-duodenal transition indicator.

61. The ingestible capsule device according to claim 50, wherein the ingestible capsule device further comprises an accelerometer, and the process further comprises:
obtaining data representing a time series of readings from the accelerometer;
determining the gastric-duodenal transition timing by:
detecting a gastric-duodenal transition indicator in the time series of readings from the accelerometer, determining that the gastric-duodenal transition indicator is caused by a gastric-duodenal transition by the ingestible capsule device, and determining the gastric-duodenal transition timing based on the timing of the detected gastric-duodenal transition indicator.

62. The ingestible capsule device according to claim 50, wherein
a spike in the CO2 concentration with respect to time, being a medical condition indicator spike, a gastroparesis indicator spike or a gastric-duodenal transition indicator spike, is detected by identifying:
a first period of increasing CO2 concentration at a rate of change with respect to time exceeding an increase gradient threshold, the increase gradient threshold being either predefined or calculated based on the subset of the time series of readings,
followed by a second period of decreasing CO2 concentration at a rate of change with respect to time exceeding a decrease gradient threshold, the decrease gradient threshold being either predefined or calculated based on the subset of the time series of readings,
wherein if the first period and the second period are identified, and if a duration between the identified first and second periods is below a predefined threshold, the readings representing the first period and the second period are detected as a spike.

63. The ingestible capsule device according to claim 13, wherein
a spike height threshold is applied to the first period of increasing CO2 concentration wherein a magnitude of increase in CO2 concentration represented by the first period is compared with the spike height threshold, and if the magnitude of increase does not meet the spike height threshold then the readings representing the first period and the second period are not detected as a spike.

64. The ingestible capsule device according to claim 50, wherein
a spike in the CO2 concentration with respect to time, being a medical condition indicator spike, a gastroparesis indicator spike or a gastric-duodenal transition indicator spike, is detected by identifying a local maximum feature being a singularity, discontinuity, or inflection point at more than a predefined threshold above a baseline value defined based on values preceding the feature.

65. The ingestible capsule device according to claim 50, wherein
processing the readings to detect one or more medical condition indicator spikes, includes:
determining that the ingestible capsule device has been ingested by the subject and the ingestion timing,
wherein
the ingestible capsule device houses an environmental temperature sensor to detect an environmental temperature at the ingestible capsule device, and determining that the ingestible capsule device has been ingested by the subject and the ingestion timing is by comparison of the environmental temperature represented by a signal from the environmental temperature sensor with a predefined temperature range for stomach of the subject or for the gastrointestinal tract of the subject.

66. The ingestible capsule device according to claim 50, wherein
the ingestible capsule device houses a relative humidity sensor to detect relative humidity at the ingestible capsule device, and determining that the ingestible capsule device has been ingested by the subject and the ingestion timing is by comparison of the relative humidity represented by a signal from the environmental temperature sensor with a predefined relative humidity range for stomach of the subject or for the gastrointestinal tract of the subject; wherein determining that the ingestible capsule device has been ingested and the ingestion timing is based on one or both of the relative humidity and the environmental temperature being within the respective predefined range.

67. The ingestible capsule device according to claim 52, wherein
diagnosing gastroparesis or suspected gastroparesis includes calculating a score representing likelihood of gastroparesis being present in the subject, the likelihood score being calculated with reference to the detected one or more spikes relative to one or more reference cases;
and wherein
calculating the likelihood score is performed by a machine learning algorithm pre-trained with labelled training data, training data being representations of CO2 concentration measured by ingestible capsule devices during residence in stomachs of respective training subjects, each training subject being clinically diagnosed by a medical practitioner as being gastroparesis positive or gastroparesis negative, and the training data being labelled with the clinical diagnosis of the respective subject.

68. A system comprising an ingestible capsule device comprising an ingestible indigestible bio-compatible housing;
and, within the housing:
a power source;
sensor hardware including gas sensing apparatus;
processor hardware;
memory hardware; and
a wireless data transmitter;
the system further comprising a receiver apparatus configured to obtain data representing a time series of readings from the gas sensing apparatus, the time series of readings being taken during exposure of the gas sensing apparatus to a gas mixture at the ingestible capsule device during passage of the ingestible capsule device through a gastrointestinal tract of a subject, the subject having orally ingested the ingestible capsule device, each reading having a value, the values of the readings being sensitive to CO2 concentration in the gas mixture;
the receiver apparatus being further configured to, at the receiver apparatus or by causing processing to be performed at a remote processing apparatus in data communication with the receiver apparatus:
process the readings to detect one or more medical condition indicator spikes in the CO2 concentration with respect to time, a medical condition indicator spike being a spike in the CO2 concentration with respect to time at a timing after an ingestion timing of the ingestible capsule device and preceding a gastric-duodenal transition timing of the ingestible capsule device.

69. A non-transitory computer-readable medium which, when executed by a processor cooperating with a memory, causes the processor to perform a method, the method comprising:
obtaining data representing a time series of readings from gas sensing apparatus housed within a ingestible capsule device orally ingested by a subjected, the time series of readings being taken during exposure of the gas sensing apparatus to a gas mixture at the ingestible capsule device during passage of the ingestible capsule device through a gastrointestinal tract of the subject, each reading having a value, the values of the readings being sensitive to CO2 concentration in the gas mixture;
processing the readings to detect one or more medical condition indicator spikes in the CO2 concentration with respect to time, a medical condition indicator spike being a spike in the CO2 concentration with respect to time at a timing after an ingestion timing of the ingestible capsule device and preceding a gastric-duodenal transition timing of the ingestible capsule device.
